(19) United States
(12) Patent Application Publication
(10) Pub. No.: US 2025/0329472 A1
(43) Pub. Date: Oct. 23, 2025

(54) SYSTEMS, METHODS, SOFTWARE, AND PLATFORMS FOR CLINICAL DECISION SUPPORT

(71) Applicant: Triaj, Inc., Annapolis, MD (US)

(72) Inventors: Martin EICHELBERGER, Annapolis, MD (US); Jane BALL, Havre de Grace, MD (US); Leroy MATTINGLY, Overland Park, KS (US); Edward McNeely ROBINSON, Kyle, TX (US); Laura Suzanne WHITEHEAD, Rockledge, FL (US); Keith ALEXANDER, Port St. Lucie, FL (US); Theodore William BENDIXSON, Earlysville, VA (US); Jonathan LEHR, South Riding, VA (US); Todd EICHELBERGER, Carlsbad, CA (US)

(21) Appl. No.: 19/184,633

(22) Filed: Apr. 21, 2025

Related U.S. Application Data
(63) Continuation of application No. 17/721,338, filed on Apr. 14, 2022, now abandoned.
Application No. 17/721,338 is a continuation of application No. PCT/US2020/055626, filed on Oct. 14, 2020.
(60) Provisional application No. 62/914,911, filed on Oct. 14, 2019.
(60) Provisional application No. 63/078,117, filed on Sep. 14, 2020.

(51) Int. Cl. G16H 70/20; G16H 10/65; G16H 20/00

(52) U.S. Cl. CPC G16H 70/20; G16H 10/65; G16H 20/00

(57) ABSTRACT

Disclosed herein are systems, methods, software, and platforms for generating and associating an identifier for an individual. Also disclosed herein are systems, methods, software, and platforms for generating personalized and customized suggestions for caring and treating the individual based on the identifier of the individual.

### CROSS-REFERENCE

This application is a continuation of U.S. application Ser. No. 17/721,338, filed on Apr. 14, 2022, which is a continuation of International Application Number PCT/US2020/055626, filed on Oct. 14, 2020, which claims the benefit of U.S. Provisional Applications: Ser. No. 62/914,911 filed on Oct. 14, 2019; and Ser. No. 63/078,117 filed on Sep. 14, 2020, the entireties of which are hereby incorporated by reference herein.

### BACKGROUND

Providing point-of-care assessment, diagnosis, and treatment is one of the foundations of modern medicine. An individual suffering from a sudden bout of symptom stemmed from injury, wound, disease, or exposure to environmental toxin must be examined and treated by healthcare professionals in a manner that is both timely and accurate. As the individual progresses through phases of examination, treatment, operation, procedure, and recovery, healthcare professionals with different areas of expertise have to constantly generate and modify effective protocols of treatment based on the health status of the individual. Communication mistakes or negligence can occur when multiple or multiple groups of healthcare professionals are required to treat an individual.

To compound the difficulty of effectively treating an individual, it is inherently problematic for individuals who do not have medical training background to adequately communicate or indicate their health status or concerns back to the healthcare professionals. In some cases, the individuals can not be well versed in medical terms. In other instances, the individuals can have limited understanding or ability to communicate with the healthcare professionals. For example, the individuals can be children or adolescents or can have limited cognitive capability. Accordingly, there remains a pressing need for systems, methods, software, and platforms to enable individuals with varying degrees of medical background and healthcare professionals to communicate, manage, and analyze medical information. Also, there remains a need to generate personalized suggestions or protocols for treating the afflicted individuals.

### SUMMARY

Described herein are systems, methods, software, and platforms for delivering a patient care plan and tracking the execution of said patient care plan. In some embodiments, a patient care plan begins with a suggestion of a presumptive diagnosis for a patient. In some embodiments, a different patient care plan is provided to different healthcare professionals. In some embodiments, a different patient care plan is provided at different stages of a patient's care.

### Traditional Care Is Typically Not Current and Not Uniform

Typically, in both in-patient and out-patient care settings, health care providers each make diagnostic and treatment decisions on an individual and discretionary basis. That is, healthcare professionals or healthcare providers typically diagnose and treat patients primarily in accordance with their own training and experience. This typical approach is not optimal due to a number of reasons. First off, diagnoses and treatments provided in the typical approach are often not associated with evidence based research or even current data. That is, healthcare is constantly changing and developing and healthcare professionals or healthcare providers who rely solely on their training and experience tend to not provide care that is current and/or that is evidence based. That is, in extreme situations, it is possible for healthcare professionals or healthcare providers to deliver care that has been demonstrated by evidence based research to not be effective or to even be detrimental to a patient.

In addition, typically there is no uniformity of care provided by healthcare professionals or healthcare providers who practice in the typical manner. This is true even in the same institution, where patients with the exact same diagnosis can receive entirely different care from different healthcare professionals or healthcare providers.

### Traditional Care Is Typically Not Provided Effectively Across Different Stages of Care

Additionally, patients in both in-patient and out-patient settings typically experience different phases or stages of care for the same condition and often interact with different healthcare professionals or healthcare providers during each phase or stage of their care.

Typically, during these different phases or stages of care there is no or minimal carry-over of treatment plans from one phase or stage to the next as healthcare professionals or healthcare providers in each stage often execute their own healthcare plan without specifically considering how to integrate their plans with those carried out by other healthcare professionals or healthcare providers. This is true of healthcare professionals or healthcare providers in different phases or stages as well as with multiple healthcare professionals or healthcare providers who provide care during the same. That is, often times an ER physician does not consider the treatment plan of a surgeon and vice versa (i.e. where the ER care and surgical care comprise different stages of care). Similarly, often times a nurse does not consider the treatment plan of a physician taking care of a patient in an in-patient setting and vice versa.

### Traditional Care Is Typically Not Closely Tracked

Because much of traditional care is carried out at the discretion of individual healthcare professionals or healthcare providers, there is typically no effective way to precisely track healthcare delivery and associated outcomes. That is, because healthcare professionals or healthcare providers can at their own discretion modify a diagnosis and/or healthcare plan, it becomes difficult to track these changes across healthcare systems, especially when the changes are not necessarily in accordance with the diagnosis or treatment plan that was initially applied.

### Systems, Methods, Software and Platforms Described Herein

In contrast to traditional, described herein are systems, methods, software, and platforms configured to provide current, uniform, and monitored healthcare to patients throughout different stages of their care.

Described herein, in some embodiments, is a system for treating an individual comprising: (a) a first non-transitory computer readable medium encoded with first software configured to cause a first processor to: (i) receive an identifier for said individual; (ii) associate said identifier with a first phase of a plurality of phases of a medical care encounter experienced by said individual; (iii) provide, to a first user, a first suggestion for caring for said individual during said first phase of said plurality of phases of said medical care encounter experienced by said individual; and (b) a second non-transitory computer readable medium encoded with second software configured to cause a second processor to: (i) receive said identifier for said individual; (ii) associate said identifier with a second phase of a plurality of phases of a medical care encounter experienced by said individual; (iii) provide, to a second user, a second suggestion for caring for said individual during said second phase of said plurality of phases of said medical care encounter experienced by said individual; wherein said plurality of phases are selected from a group consisting of: an assessment phase, a diagnostic phase, a resuscitation phase, a pre-procedure phase, a procedure phase, a post-procedure phase, critical care phase, after care phase, or discharge. Described herein, in some embodiments, is a system for treating an individual comprising: (a) a first non-transitory computer readable medium encoded with first software configured to cause a first processor to: (i) receive an identifier for said individual; (ii) associate said identifier with a first phase of a plurality of phases of a medical care encounter experienced by said individual; (iii) provide, to a first user, a first suggestion for caring for said individual during said first phase of said plurality of phases of said medical care encounter experienced by said individual; and (b) a second non-transitory computer readable medium encoded with second software configured to cause a second processor to: (i) receive said identifier for said individual; (ii) associate said identifier with a second phase of a plurality of phases of a medical care encounter experienced by said individual; (iii) provide, to a second user, a second suggestion for caring for said individual during said second phase of said plurality of phases of said medical care encounter experienced by said individual; wherein said plurality of phases are selected from a group consisting of: an assessment phase, a diagnostic phase, a resuscitation phase, a pre-operation phase, an operation phase, a post-operation phase, critical care phase, after care phase, or discharge. In some embodiment, the first processor or the second processor is a component of a mobile computing device. In some embodiments, the first processor or the second processor is a component of a desktop computer. In some embodiments, the first software or the second software is an application (App.). In some embodiments, the first non-transitory medium or the second non-transitory medium is a component of a remote server. In some cases, the identifier for said individual comprises a name or medical record number of said individual. In some embodiment, the first processor or the second processor is a component of a mobile computing device. In some embodiments, the first processor or the second processor is a component of a desktop computer. In some embodiments, the first software or the second software is an application (App.). In some embodiments, the first non-transitory medium or the second non-transitory medium is a component of a remote server. In some cases, the identifier for said individual comprises a name or medical record number of said individual. In some instances, the system further comprises a barcode reader and said software is configured to cause said first processor to receive said identifier from said barcode reader. In some embodiments, said first user or said second user comprises a healthcare professional selected from the group consisting of a physician, an emergency medical technician, a nurse, a paramedic, a nurse practitioner, a physician's assistant, a nurse's aide, and an x-ray technician. In some cases, said first user or said second user is not a healthcare provider. In some embodiments, said first user is also said individual. In some cases, said first suggestion or said second suggestion comprises a diagnosis. In some embodiments, said first suggestion or said second suggestion comprises at least one of a therapeutic, a dosage of said therapeutic, or a timing of delivery of said therapeutic. In some embodiments, said first suggestion or said second suggestion comprises a protocol. In some embodiments, said protocol is a hospital protocol. In some cases, said protocol is customized to said individual. In some instances, said protocol is an evidence-based protocol. In some embodiments, said first software is configured further cause said first processor to receive input from said first user indicating that said first user completed at least a portion of said protocol. In some embodiments, said first suggestion or said second suggestion is specific to each phase. In some embodiments, said first suggestion is specific to said first user. In some embodiments, said first software is configured to further cause said first processor to receive input from said first user which at least partially determines said second suggestion. In some embodiments, said first software or said second software is configured to further cause said first processor or said second processor to receive data from an electronic medical record of said individual. In some embodiments, said first suggestion or said second suggestion is based at least in part on said data. In some embodiments, said data indicates whether said first user applied said first suggestion to said first phase and second user applied said second suggestion to said second phase. In some embodiments, the system tracks an outcome of said medical care encounter. In some embodiments, said first suggestion is available to said second user. In some embodiments, said procedure or operation phase comprises a surgical procedure or a surgical operation.

Described herein, in some instances, is a computer implemented method for treating an individual, comprising: receiving an identifier for said individual; associating said identifier with each phase of a plurality of phases of a medical care encounter experienced by said individual; and providing, to a user, a suggestion for caring for said individual during each phase of said plurality of phases of said medical care encounter experienced by said individual; wherein said plurality of phases are selected from a group consisting of: an assessment phase, a diagnostic phase, a resuscitation phase, a pre-procedure phase, a procedure phase, a post-procedure phase, critical care phase, after care phase, or discharge. Described herein, in some instances, is a computer implemented method for treating an individual, comprising: receiving an identifier for said individual; associating said identifier with each phase of a plurality of phases of a medical care encounter experienced by said individual; and providing, to a user, a suggestion for caring for said individual during each phase of said plurality of phases of said medical care encounter experienced by said individual; wherein said plurality of phases are selected from a group consisting of: an assessment phase, a diagnostic phase, a resuscitation phase, a pre-operation phase, am operation phase, a post-operation phase, critical care phase, after care phase, or discharge. In some embodiments, said identifier for said individual comprises a name or medical record number of said individual. In some embodiments, the method comprises receiving said identifier from a barcode reader. In some embodiments, said user comprises a healthcare professional selected from the group consisting of a physician, an emergency medical technician, a nurse, a paramedic, a nurse practitioner, a physician's assistant, a nurse's aide, and an x-ray technician. In some embodiments, said user is not a healthcare provider. In some embodiments, said user is also said individual. In some embodiments, said suggestion comprises a diagnosis. In some embodiments, said suggestion comprises at least one of a therapeutic, a dosage of said therapeutic, or a timing of delivery of said therapeutic. In some embodiments, said suggestion comprises a protocol. In some embodiments, the protocol is a hospital protocol. In some embodiments, said protocol is customized to said individual. In some embodiments, said protocol is an evidence-based protocol. In some embodiments, the method comprises receiving input from said user indicating that said user completed at least a portion of said protocol using said processor. In some embodiments, said suggestion is specific to each phase. In some embodiments, said suggestion is specific to said user. In some embodiments, the method comprises providing to said user a first suggestion in a first phase and a different user a second suggestion in a second phase. In some embodiments, the method comprises receiving input from said first user which at least partially determines said second suggestion. In some embodiments, the method comprises a software configured to further cause said processor to receive data from an electronic medical record of said individual. In some embodiments, said suggestion is based at least in part on said data. In some embodiments, said data comprises an indication of whether said user applied said suggestion to said medical care encounter. In some embodiments, the method comprises tracking an outcome of said medical care encounter. In some embodiments, the method comprises tracking whether said suggestion is followed at each of said plurality of phases. In some embodiments, said suggestion is available to said user and a different user. In some embodiments, said procedure phase comprises a surgical procedure. In some embodiments, said operation phase comprises a surgical operation.

Described herein, in some embodiments, is a platform for treating an individual comprising: (a) a first user portal on a first computing device configured to: receive an identifier for said individual; associate said identifier with a first phase of a plurality of phases of a medical care encounter experienced by said individual; and provide, to a first user, a suggestion for caring for said individual during said first phase of said plurality of phases of said medical care encounter experienced by said individual; and (b) a second user portal on a second computing device configured to: provide, to a second user, a suggestion for caring for said individual during a second phase of said plurality of phases of said medical care encounter experienced by said individual; wherein said plurality of phases are selected from a group consisting of: an assessment phase, a diagnostic phase, a resuscitation phase, a pre-procedure phase, a procedure phase, a post-procedure phase, critical care phase, after care phase, or discharge. Described herein, in some embodiments, is a platform for treating an individual comprising: (a) a first user portal on a first computing device configured to: receive an identifier for said individual; associate said identifier with a first phase of a plurality of phases of a medical care encounter experienced by said individual; and provide, to a first user, a suggestion for caring for said individual during said first phase of said plurality of phases of said medical care encounter experienced by said individual; and (b) a second user portal on a second computing device configured to: provide, to a second user, a suggestion for caring for said individual during a second phase of said plurality of phases of said medical care encounter experienced by said individual; wherein said plurality of phases are selected from a group consisting of: an assessment phase, a diagnostic phase, a resuscitation phase, a pre-operation phase, am operation phase, a post-operation phase, critical care phase, after care phase, or discharge. In some embodiments, said first user portal or said second user portal are a component of an App. In some embodiments, said identifier for said individual comprises a name or medical record number of said individual. In some embodiments, said first user or said second user comprises a healthcare professional selected from the group consisting of a physician, an emergency medical technician, a nurse, a paramedic, a nurse practitioner, a physician's assistant, a nurse's aide, and an x- ray technician. In some embodiments, said first user or said second user is not a healthcare provider. In some embodiments, said first user is also said individual. In some embodiments, said first suggestion or said second suggestion comprises a diagnosis. In some embodiments, said first suggestion or said second suggestion comprises at least one of a therapeutic, a dosage of said therapeutic, or a timing of delivery of said therapeutic. In some embodiments, said first suggestion or said second suggestion comprises a protocol. In some embodiments, said protocol is a hospital protocol. In some embodiments, said protocol is customized to said individual. In some embodiments, said protocol is an evidence-based protocol. In some embodiments, the platform comprises a first software is configured further cause said first processor to receive input from said first user indicating that said first user completed at least a portion of said protocol. In some embodiments, said first suggestion or said second suggestion is specific to each phase. In some embodiments, said first suggestion is specific to said first user. In some embodiments, said first software is configured to further cause said first processor to receive input from said first user which at least partially determines said second suggestion. In some embodiments, said first user portal or said second user portal is configured to receive data from an electronic medical record of said individual. In some embodiments, said first suggestion or said second suggestion is based at least in part on said data. In some embodiments, said data indicates whether said first user applied said first suggestion to said first phase and second user applied said second suggestion to said second phase. In some embodiments, the platform tracks an outcome of said medical care encounter is tracked. In some embodiments, said first suggestion is available to said second user. In some embodiments, said procedure phase comprises a surgical procedure. In some embodiments, said operation phase comprises a surgical operation.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments and the accompanying drawings.

FIG. 1 illustrates an exemplary organization or a medical care facility where the plurality of phases of caring and treating an individual occurs. Healthcare professionals can treat and care for the individual in one or more of the departments or organizational compartments of the medical care facility.

FIG. 2 illustrates an exemplary procedure and operation checklist structure that forms the basis of the plurality of phases of medical care encounter and the accompanying treatments. Each oval represents a phase or treatment where suggestions can be provided. The interconnecting lines represent clustering of the suggestions provided by the phase or treatment as determined by the systems, methods, software, and platforms as described herein.

FIG. 3 shows a non-limiting example of a computing device; in this case, a device with one or more processors, memory, storage, and a network interface;

FIG. 4 shows a non-limiting example of a web/mobile application provision system; in this case, a system providing browser-based and/or native mobile user interfaces; and

FIG. 5 shows a non-limiting example of a cloud-based web/mobile application provision system; in this case, a system comprising an elastically load balanced, auto-scaling web server and application server resources as well synchronously replicated databases.

FIG. 6 illustrates exemplary screen captures of graphic user interface of a web application displayed on a computing device, where the users interact and utilize the systems, methods, software, and platforms as described herein to create a patient profile to treat and care for an individual. FIG. 6A illustrates the graphic user interface of a patient session organized in the web application. FIG. 6B illustrates a patient profile as part of an identifier associated with the individual being treated, where the patient profile comprises age and weight of the patient. FIG. 6C illustrates assigning a patient profile based on the inputs from FIG. 6B. FIG. 6D illustrates selection of mechanisms of injury and the accompanying diagnosis. FIG. 6E illustrates a demonstration video directed to the a mechanism of injury of side impact motor vehicle crash. FIG. 6F illustrates a phase of care selection. FIG. 6G illustrates the selection of resuscitation for the phase of care. FIG. 6H illustrates the profile page of the patient who suffers from side impact from motor vehicle crash.

FIG. 7 illustrates exemplary screen captures of graphic user interface of a web application displayed on a computing device, where the users interact and utilize the systems, methods, software, and platforms as described herein to complete a patient profile to treat and care for an individual by adding diagnosis, operation, or procedure allergies, and/or medical history. FIG. 7A illustrates a checklist for the patient who is being treated. FIG. 7B illustrates screen shots for diagnosis selection of a femur fracture for the checklist. FIG. 7C illustrates operation or procedure selection based on the femur fracture selection from FIG. 7B. FIG. 7D illustrates selection of allergies (FIG. 7E) and medical history (FIG. 7F) prior to the operation or procedure.

FIG. 8 illustrates exemplary screen captures of graphic user interface of a web application displayed on a computing device, where the users interact and utilize the systems, methods, software, and platforms as described herein to treat and care for an individual via checklist navigation. FIG. 8A-C illustrate primary survey selection, where a tourniquet is selected based on the input (FIG. 8D). FIG. 8E illustrates procedures directed to application of tourniquet. FIG. 8F illustrates instruction to confirm absence of external hemorrhage. FIG. 8G illustrates instruction to confirm that the patient's airway is not obstructed. FIG. 8H illustrates completion of the checklist of the web application.

FIG. 9 illustrates exemplary screen captures of graphic user interface of a web application displayed on a computing device, where the users interact and utilize the systems, methods, software, and platforms as described herein for searching procedures or operations to treat and care for an individual. FIG. 9A-C illustrate procedure or operation search selection, where blood product administration is chosen and displayed. FIG. 9D illustrates completion of the procedure or operation search.

FIG. 10 illustrates exemplary screen captures of graphic user interface of a web application displayed on a computing device, where the users interact and utilize the systems, methods, software, and platforms as described herein to prioritize a patient session to treat and care for an individual. FIG. 10A illustrates a completed patient profile. FIG. 10B illustrates multiple patient session profiles. FIG. 10C illustrates a patient session being selected for deletion, and FIG. 10D illustrates confirmation of a patient session being deleted.

FIG. 11 illustrates how the suggestions are provided based on the input of the individual suffering from a femur fracture as described in FIG. 7. FIG. 11A provides an exemplary flowchart of providing suggestions for treating a closed femur fracture as determined by the systems, methods, software, and platforms as described herein. FIG. 11B provides an exemplary checklist or protocol generated by the systems, methods, software, and the platforms as described in the instant disclosure for treating the individual is the femur fracture is a open fracture.

FIG. 12 illustrates a work flow of a first user and a second user utilizing the systems, methods, software, and platforms described herein to generate suggestions for treating the individual.

FIG. 13 illustrates a specific example of healthcare professionals utilizing the systems, methods, software, and platforms described herein to treat an individual who is a child experiencing a medical emergency.

### DETAILED DESCRIPTION

While various embodiments have been shown and described herein, it will be obvious to those skilled in the art that such embodiments are provided by way of example only. Numerous variations, changes, and substitutions can occur to those skilled in the art without departing from the invention. It should be understood that various alternatives to the embodiments described herein can be employed. Aspects, features and advantages of exemplary embodiments will be better understood with regard to the following description in connection with the accompanying drawings. It should be apparent to those skilled in the art that the described embodiments provided herein are illustrative only and not limiting, having been presented by way of example only. All features disclosed in this description can be replaced by alternative features serving the same or similar purpose, unless expressly stated otherwise. Therefore, numerous other embodiments of the modifications are contemplated as falling within the scope of the present disclosure as defined herein and equivalents thereto. Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Any systems, methods, software, and platforms described herein are modular and not limited to sequential steps. Accordingly, terms such as “first” and “second” do not necessarily imply priority, order of importance, or order of acts.

### Overview

The present disclosure relates to systems, methods, software, and platforms: receiving an identifier from an individual; associating the identifier with a first phase of a plurality of phases of the medical care encounter experienced by the individual; providing suggestions for caring and treating the individual during the first phase of the plurality of phases of the medical care encounter experienced by the individual; associating the identifier with a second phase of the plurality of phases of the medical care encounter; and providing suggestions for caring or treating the individual during the second phase of the plurality of phases of the medical care encounter experienced by the individual. In some embodiments, the plurality of phases of the medical care encounter for the individual comprises an assessment phase, a diagnostic phase, a resuscitation phase, a pre-procedure phase, a procedure (or surgery) phase, a post-procedure phase, critical care phase, after care phase, or discharge. In some embodiments, the plurality of phases of the medical care encounter for the individual comprises an assessment phase, a diagnostic phase, a resuscitation phase, a pre-operation (or pre-op) phase, an operation (or surgery) phase, a post-operation (or post-op) phase, critical care phase, after care phase, or discharge.

In some cases, the systems, methods, software, and platforms utilize non-transitory computer readable mediums, software, and processors. In some instances, the processors comprise components of mobile computing device, desktop computer, or any other computing devices as disclosed herein. In some embodiments, the processors are configured to send, receive, store, and analyze the identifier of the individual. In some embodiments, the processors as described herein comprise a first processor, a second processor, and any other additional processors. In some cases, the first processor: receives the identifier of the individual; associates the identifier with a first phase of the plurality of phase of the medical care encounter experienced by the individual; and provides a first suggestion for treating and caring for the individual during the first phase of the plurality of phases of the medical care encounter experienced by the individual. In some embodiments, the second processor: receives the identifier of the individual; associates the identifier with a second phase of the plurality of phase of the medical care encounter experienced by the individual; and provide a second suggestion for treating and caring for the individual during the second phase of the plurality of phases of the medical care encounter experienced by the individual. In some instances, the second processor provides the second suggestion based on the first suggestion.

In some cases, the software as described herein comprises program, algorithm, web application, or mobile application. In some instances, the software comprises a first software, a second software, or additional software configured to receive the identifier, associates the identifier with plurality of phases of the medical care encounter, and provides suggestions for each of the phases of the medical care encounter. In some embodiments, the first software provides the second software with suggestions based on the identifier and input received by the first software. In some embodiments, the first software determines the suggestions for the second software. In some embodiments, the first software partially determines the suggestions for the second software. In some embodiments, the first and second software can be a web application or a mobile application.

In some embodiments, the non-transitory computer readable mediums as described herein comprise components of a server. In some cases, the server is a remote server. In some embodiments, the systems, methods, software, and platforms as described herein comprise utilizing additional components such as barcode readers, vital sign scanners, or any other medical devices, apparatus, and equipment. In some embodiments, the server determines the first or second suggestions. In some embodiments, the server determines at least one suggestion for at least one phase of the plurality of phases of medical care encounter.

In some embodiments, the systems, methods, software, and platforms as described herein are used by a first user, a second user, or additional users. In some cases, the first user is the individual with the identifier and is being treated. In some embodiments, the first user is a healthcare professional. In some embodiments, the second user is not a healthcare professional. In some embodiments, the second user is a healthcare professional. In some cases, the first user is the individual who is not a healthcare professional, and the second user is the healthcare professional. In some embodiments, both the first and second users are healthcare professionals. Non-limiting examples of healthcare professionals include a physician, an emergency medical technician (EMT), a nurse, a paramedic, a nurse practitioner, a physician's assistant, a nurse's aide, an x-ray technician, a pharmacist, a physical therapist, a social worker, a dietician, an information technology technician, a pathologist, a radiologist, a clinical researcher, a laboratory technician, military corpsman or any military and veteran healthcare professionals or veteran healthcare providers, or any staff performing services in administration, information technology, therapeutics, diagnostics, social services, child protective services, psychiatry, psychology, children welfare, and support in a medical care facility.

In some cases, the provided suggestions comprise diagnosis, protocols, instructions, illustration, warning, or checklist for administering treatments during each phase of the plurality phases of the medical care encounter. In some embodiments, the provided suggestions comprise suggestions for therapeutics, dosages of the therapeutics, timing and regiment of the therapeutics, and route of delivery of the therapeutics. In some cases, the protocols are hospital protocols. In some embodiments, the protocols are evidence-based protocols. In some embodiments, the protocols are personalized or customized to the individual experiencing the plurality of medical care encounter. In some embodiments, the protocols are personalized or customized based on the identifier of the individual. In some embodiments, the suggestions comprise first suggestions, second suggestions, or any additional suggestions, where each of the suggestions is independently specific to the phases of the plurality phases of the medical care encounter. In some embodiments, the first suggestion determines the second suggestion. In some embodiments, the first suggestion partially determines the second suggestion. In some embodiments, the provided suggestions, based on the analysis of the identifier, can be modified, added, removed, shifted, analyzed, overridden, suspended, recorded, monitored, and tracked by the healthcare professionals. In some embodiments, the provided suggestions are modular and can be interchanged based on the identifier at any time of any phase of the plurality of phases of the medical care encounter. In some embodiments, the first suggestion and the second suggestion can be independently specific to any one of the phases. In some instances, the first suggestion and the second suggestion can be associated with multiple phases. In some embodiments, the first suggestion, second suggestion, and any additional suggestions are not sequential and can be associated with any of the phases in any order.

In some instances, the systems, methods, software, and platforms as described herein comprise a first user portal, a second user portal, or other additional user portals. In some instances, the first user portal comprises a first computing device configured: to receive the identifier of the individual; to associate the identifier with a first phase of a plurality of phases of a medical care encounter experienced by the individual; and to provide to the first user a first suggestion for caring or treating the individual during the first phase of the plurality of phases of the medical care encounter experienced by the individual. In some embodiments, the second user portal comprises a second computing device configured: to receive the identifier of the individual; to associate the identifier with a second phase of a plurality of phases of a medical care encounter experienced by the individual; and to provide to the second user a second suggestion for caring or treating the individual during the second phase of the plurality of phases of the medical care encounter experienced by the individual. In some embodiments, the second suggestion is at least partially determined by the first suggestion.

### Phases of a Medical Care Encounter

In some instances, as described herein are systems, methods, software, and platforms that receive an identifier of an individual who is experiencing a plurality phases of a medical care encounter. Also described herein are systems, methods, software, and platforms associating the identifier with a first, a second, or any other phases of the plurality of phases. In some embodiments, suggestions are provided for treating or caring for the individual during the first, the second, or any other phases of the plurality phases of medical encounter. In some embodiments, the plurality of phases of medical care encounter includes, but not limited to, an assessment phase, a diagnostic phase, a resuscitation phase, a pre-procedure phase, a pre-operation phase, a procedure (or surgery) phase, an operation phase, a post-procedure phase, a post-operation phase critical care phase, after care phase, or discharge. In some cases, healthcare professionals can care and treat the individual in any one of the phases. In some cases, the same healthcare professionals can care and treat the individual in at least one of the phases. In some instances, the same healthcare professionals can care and treat the individual in multiple phases. In some instances, the same healthcare professionals can care and treat the individual in multiple continuous phases.

In some instances, certain healthcare professionals care for or treat the individual in at least one assigned phase. That is, in these embodiments, a healthcare professional treats a patient during a specific phase of treatment but not in any other phase of treatment.

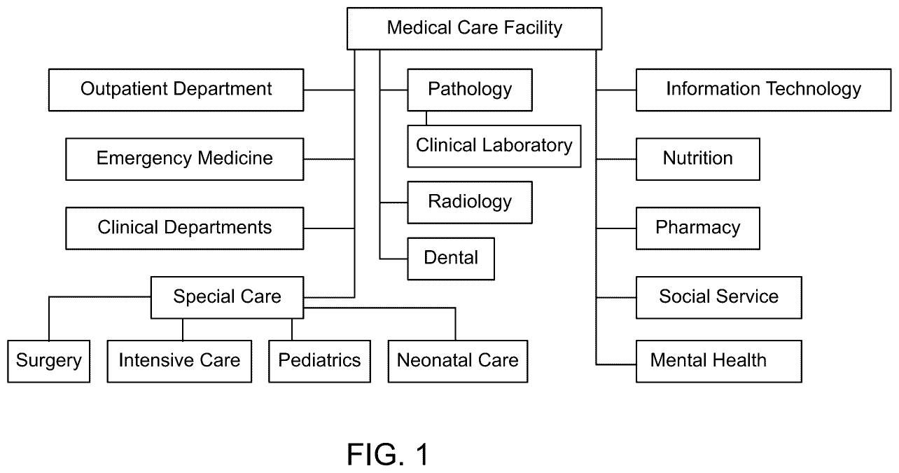

In some embodiments, the individual being treated and cared for does not have to undergo every phase of the plurality of phases of a medical care encounter. In some cases, the individual does not have to undergo the plurality of phases in a sequential manner. In some cases, the individual can undergo any of the phases more than once. In some embodiments, the phases of the medical care encounter experienced by the individual can be changed at any time based on the status of the individual. In some cases, the phases of the medical care encounter comprise healthcare professionals from any divisions or departments of a medical care facility. Healthcare professionals can treat or care for the individual in any or in multiple phases of the plurality of phases of the medical care encounter. Furthermore, the individual phases of a medical care encounter can each comprise treatments, tests, procedures, and/or operations that are interlinked. FIG. 1 illustrates an exemplary organizational chart of healthcare professionals in a medical care facility. Non-limiting examples of medical care facilities can include hospitals, field hospital, medical transport, military medical installations, ambulatory surgical centers, birth centers, urgent care clinics, nursing homes, long term care facilities, medical offices, dental offices, clinics, optical centers, clinical laboratories, home healthcare, hospice homes, dialysis centers, imaging and radiology enters, mental health and addiction treatment centers, or orthopedic and other rehabilitation centers.

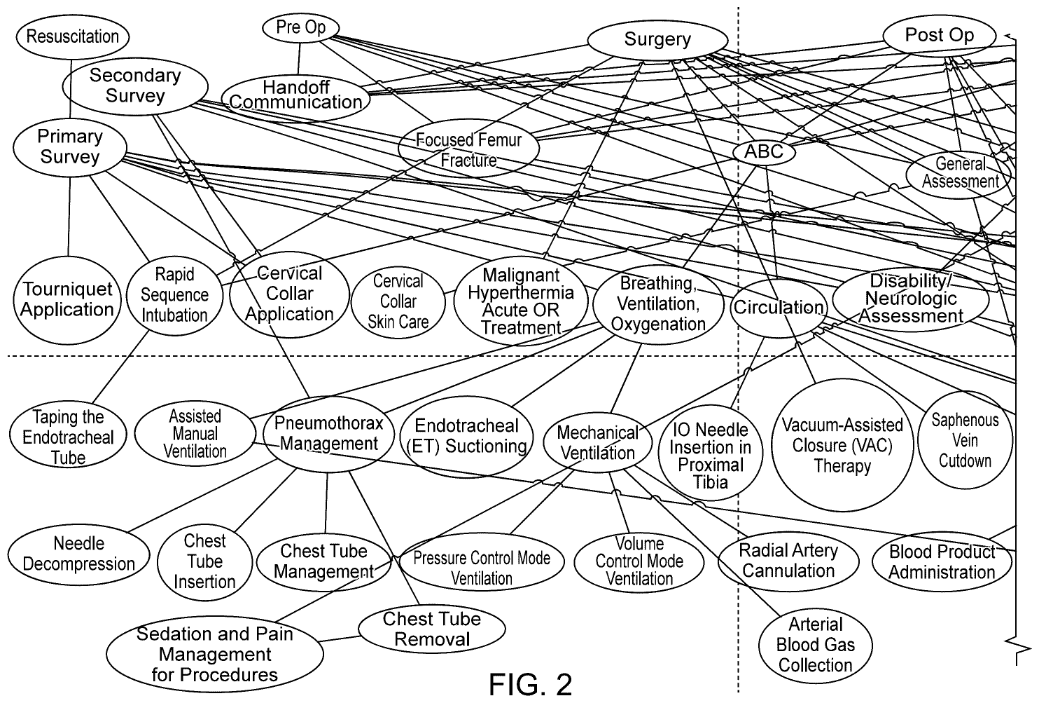

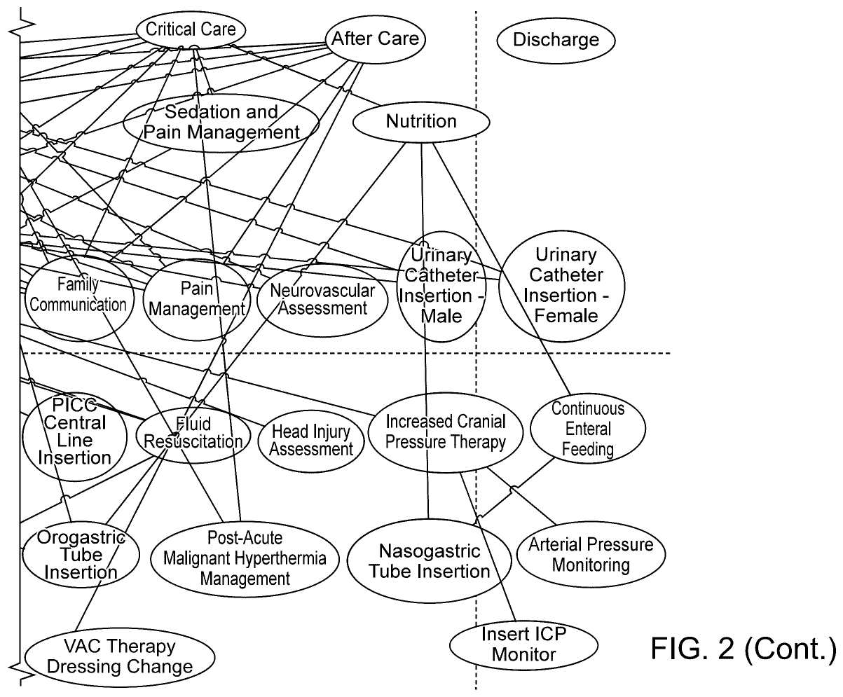

Healthcare professionals can be part of any of departments or organizational compartments of the medical care facility. In some embodiments, the plurality of phases can involve any of the departments or organizational compartments. FIG. 2 further demonstrates an exemplary flow chart of a procedure and operation checklist structure where procedures or operations, in some instances, are performed in sequential manner or independent of one another. Exemplary phases of the plurality of phases and the accompanying treatments as depicted by FIG. 2 include resuscitation, primary survey, secondary survey, pre-procedure (pre-op) handoff communication, procedure (surgery), post-procedure (post-op), critical care, after care, discharge, nutrition, sedation and pain management, general assessment, airway, breathing, and circulation (ABC), focused femur fracture, tourniquet application, rapid sequence intubation, cervical cellar application, cervical collar skin care, malignant hyperthermia (acute), breathing, ventilation, and oxygenation, circulation, disability neurologic assessment, family communication, pain management, neurovascular assessment, urinary catheter insertion (male or female), taping the endotracheal tube, assisted manual ventilation, pneumothorax management, endotracheal (ET) suctioning, mechanical ventilation, intraosseous (IO) needle insertion in proximal tibia, vacuum-assisted closure (VAC) therapy, saphenous vein cutdown, peripherally inserted central venous catheter (PICC central line) insertion, fluid resuscitation, head injury assessment, increased cranial pressure therapy, continuous internal feeding, needle decompression, chest tube insertion, chest tube management, chest tube removal, sedation and pain management for procedures or surgeries, pressure control mode ventilation, volume control mode ventilation, arterial blood gas collection, radial artery cannulation, blood product administration, orogastric tube insertion, post-acute malignant hyperthermia management, nasogastric tube insertion, insertion of intracranial pressure monitor, arterial pressure monitoring; or negative-pressure wound therapy dressing change (VAC therapy dressing change). Also demonstrated in FIG. 2, some of the phases and the treatment options are intentionally clustered together, as shown by the interconnecting lines, to provide suggestions for the most effective treatment and care for the individual. In some cases, the phases and treatment options are weighted based on machine learning algorithms for the clustering. Accordingly, the plurality of phases of medical care encounter, the healthcare professionals involved in the medical care encounter, and the treatment options, testing, procedures, or operations utilized during the medical care encounter are highly dynamic and subject to change based on the status and response of the individual.

### Assessment

In some embodiments, the systems, methods, software, and platforms as described herein provide suggestions for assessing an individual during an assessment phase. In some embodiments the suggestions for the assessment are based on identifier of the individual. In some cases, the identifier can be generated or modified by any other phases of the plurality of phases of medical care encounter. In some embodiments, the suggestions for the assessment phase can be at least partially determined by suggestions of any other phase of the plurality of phases. In some embodiments, the suggestions for the assessment phase for treating or caring for the individual is provided at a point-of-care (POC) capacity. In some cases, the suggestions of assessment phase comprises protocols instructing healthcare professionals such as EMT to examine the scene by determining safety of the scene, utilizing body substance isolation (BSI), documenting the types of medical emergency or medical needs of the individual, and determining if additional medical resources are required or if advanced life support (ALS) needs to be administered. Exemplary protocols of ALS comprises tracheal intubation, rapid sequence induction, cardiac monitoring, cardiac defibrillation, intravenous cannulation, intraosseous access and infusion, surgical cricothyrotomy, needle cricothyrotomy, needle decompression of tension pneumothorax, administering medication through parenteral and enteral routes, advanced cardiac life support, pediatric advanced life support, and pre-hospital trauma life support. In some cases, the suggestion of the assessment phase comprises protocols for the healthcare professionals identifying and triaging life-threating medical issues. In some cases, emphasis is placed on the individual's level of consciousness, cervical spinal stabilization, airway, breathing, and circulation. In some cases, protocols for transporting the individual are generated as part of the assessment phase.

Other non-limiting examples of suggestions provided by the systems, methods, software, and platforms include assessment of the individual's general impression, mental state, responsiveness, airway blockage, breathing, blood circulation (assessment for major bleeding, pulse, etc). In some cases, when the individual is a pediatric patient the suggestion of assessment comprise assessment of brachial pulse and capillary refill. In some cases, the suggestions can include instructions and protocols for healthcare professionals to: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians.

In some embodiments, the identifier of the individual indicates that the individual is a priority patient. As such, the identifier is transmitted to the medical care facility that would be treating the individual. Upon receiving the identifier indicating the priority patient status of the individual, suggestions provided by the systems, methods, software, and platforms can instruct the healthcare professionals to conduct focused history and physical exam and rapid trauma assessment. In some embodiments, the healthcare professionals can confirm, deny, modify, add, remove, shift, analyze, override, suspend, record, monitor, and track the suggestions of the assessment phase. In some embodiments, the suggestions of assessment phase can not be changed. In some embodiments, the suggestions of assessment phase must be followed by the healthcare professionals. In some embodiments, certain healthcare professionals can examine and overrule suggestions of other healthcare professionals. In some embodiments, healthcare professionals can examine and verify whether other healthcare professionals are adhering to the suggestions. For example, physician at emergency room can examine and override suggestions based on initial assessment of EMT.

In some embodiments, suggestions of assessment phase and identifier generated and modified in assessment phase can at least partially determine suggestions of other phases. In some embodiments, suggestions of assessment phase are first suggestions and can at least partially determine second suggestions of a second phase. In some embodiments, suggestions of assessment phases are second suggestions and can be partially determined by first suggestions of a first phase. In some cases, first and second phases can be the same or different. In some cases, suggestions of assessment phase can be both first and second suggestions. In some embodiments, suggestions for assessment are first suggestions and provided to a first user for assessing the individual during a first phase of assessment. In some cases, the first user is the individual. In some cases, the first user is a healthcare professional. In some embodiments, the first user is not a healthcare professional. In some embodiments, suggestions for assessment are second suggestions and provided to a second user for assessing the individual during a second phase of assessment. In some instances, the second user is a healthcare professional.

### Diagnosis

In some embodiments, the systems, methods, software, and platforms as described herein provide suggestions for diagnosing individual during a diagnosis phase. In some embodiments the suggestions for the diagnosis are based on identifier of the individual. In some cases, the identifier has been generated or modified in the assessment phase. In some instances, the suggestions are based on the identifier that has been generated and modified during any of the plurality of phases of the medical care encounter experienced by the individual. In some embodiments, the suggestions of the diagnosis phase can be at least partially determined by suggestions of any of the plurality of phases of medical care encounter.

In some embodiments, the suggestions of the diagnosis phase instruct the healthcare professionals to conduct diagnostic procedures based on general diagnostic procedure, differential diagnosis, pattern recognition, criteria, clinical diagnosis, laboratory diagnosis, radiology diagnosis, tissue diagnosis, principle diagnosis, admitting diagnosis, prenatal diagnosis, diagnosis of exclusion, dual diagnosis, self-diagnosis, remote diagnosis, nursing diagnosis, and computer-aided diagnosis. Exemplary diagnosis suggested by the systems, methods, software, and platforms as described herein include lab test, biopsy test, blood test, stool test, urinalysis, cardiac stress test, electrocardiogra electroencephalography, electromyography, electroneuronography, electronystagmography, electrooculography, electroretinography, endoluminal capsule monitoring, endoscopy, colonoscopy, colposcopy, cystoscopy, gastroscopy, laparoscopy, laryngoscopy, ophthalmoscopy, otoscopy, sigmoidoscopy, esophageal motility study, evoked potential, magnetoencephalography, medical imaging, angiography, aortography, cerebral angiography, coronary angiography, lymphangiography, pulmonary angiography, ventriculography, chest photofluorography, computed tomography, echocardiography, electrical impedance tomography, fluoroscopy, magnetic resonance imaging, diffuse optical imaging, diffusion tensor imaging, diffusion-weighted imaging, functional magnetic resonance imaging, positron emission tomography, radiography, scintillography, single-photon emission computed tomography, ultrasonography, contrast-enhanced ultrasound, gynecologic ultrasonography, intravascular ultrasound, obstetric ultrasonography, thermography, virtual colonoscopy, neuroimaging, or posturography. Additional diagnosis can include diagnosis for: increased intracranial pressure—unspecified, epidural hematoma, subdural hematoma, diffuse axonal injury, contusion, skull fracture, pneumothorax, abdominal injury—unspecified, pneumoperitoneum, femur fracture, compartment syndrome, open wound, hypovolemic shock, or malignant hyperthermia.

In some embodiments, the systems, methods, software, and platforms as described herein provide suggestions such as instructions and protocols for healthcare professionals to: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians.

In some embodiments, the systems, methods, software, and platforms as described herein allow healthcare professionals to confirm, deny, modify, add, remove, shift, analyze, override, suspend, record, monitor, and track the suggestions of the diagnosis phase. In some embodiments, the suggestions of diagnosis phase cannot be changed. In some embodiments, the suggestions of diagnosis phase must be followed by the healthcare professionals. In some embodiments, the suggestions of the diagnosis phase and the identifier generated and modified in the diagnosis phase can at least partially determine suggestions of any other phases. In some embodiments, suggestions of diagnosis phase are first suggestions and can at least partially determine second suggestions of a second phase. In some embodiments, suggestions of diagnosis phases are second suggestions and can be partially determined by first suggestions of a first phase. In some embodiments, first and second phases can be the same or different. In some cases, suggestions of diagnosis phase can be both first and second suggestions. In some embodiments, suggestions for diagnosis are first suggestions and provided to a first user for diagnosing the individual during a first phase of diagnosis. In some cases, the first user is the individual. In some cases, the first user is a healthcare professional. In some embodiments, the first user is not a healthcare professional. In some embodiments, suggestions for assessment are second suggestions and provided to a second user for diagnosing the individual during a second phase of diagnosis. In some instances, the second user is a healthcare professional.

### Resuscitation

In some instances, the systems, methods, software, and platforms as described herein provide suggestions for resuscitating individual during a resuscitation phase. In some embodiments, suggestions for resuscitation are based on identifier of the individual. In some instances, the identifier has been generated or modified by any other phases of the plurality of phases of medical care encounter. In some embodiments, suggestions for resuscitation phase can be at least partially determined by suggestions of any other phase of the plurality of phases. Non-limiting examples of the suggestions pertaining to resuscitating the individual comprise: checking the responsiveness of the individual; checking for signs of cardiac arrest; notifying other healthcare professionals specialized resuscitation (e.g. a resuscitation team or medical emergency team); assessing the individual's airway, breathing, circulation, disability and exposure; providing the individual with oxygen, recording vital signs, opening airway if necessary; and administering cardiopulmonary resuscitation (CPR) or defibrillator.

In some embodiments, the systems, methods, software, and platforms as described herein provide suggestions such as instructions and protocols for healthcare professionals to: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians.

In some embodiments, the healthcare professionals can confirm, deny, modify, add, remove, shift, analyze, override, suspend, record, monitor, and track the suggestions of the resuscitation phase. In some alternatives, suggestions of resuscitation phase can not be changed. In some instances, the suggestions of resuscitation phase must be followed by healthcare professionals. In some embodiments, certain healthcare professionals can examine and overrule suggestions of other healthcare professionals. In some embodiments, healthcare professionals can examine and verify whether other healthcare professionals are adhering to the suggestions. In some embodiments, suggestions of resuscitation phase and identifier generated and modified in resuscitation phase can at least partially determine suggestions of any other phases. In some embodiments, suggestions of resuscitation phase are first suggestions and can at least partially determine second suggestions of a second phase. In some embodiments, suggestions of resuscitation phases are second suggestions and can be at least partially determined by first suggestions of a first phase. In some cases, suggestions of resuscitation phase can be both first and second suggestions. In some embodiments, suggestions for resuscitation are first suggestions and provided to a first user resuscitating the individual during a first phase of resuscitation. In some instances, the first user is a healthcare professional. In some cases, the first user is not a healthcare professional. In some embodiments, suggestions for resuscitation are second suggestions and provided to a second user resuscitating the individual during a second phase of resuscitation. In some instances, the second user is a healthcare professional.

### Pre-Procedure

In some embodiments, the systems, methods, software, and platforms as described herein provide suggestions for healthcare professionals to care for the individual prior to performing a procedure to the individual. In some alternatives, suggestions for the pre-procedure are based on identifier of the individual. In some cases, identifier has been generated or modified by any other phases of the plurality of phases of medical care encounter. In some embodiments, suggestions for pre-procedure phase can be at least partially determined by suggestions of any other phase of the plurality of phases. In some instances, the suggestions instruct verifying the procedure with both the individual and the healthcare professionals. In some instances, the verification comprises correct identity of the individual, correct procedure type, correct site of procedure, and correct items for the procedure. In some embodiments, the suggestions comprise a checklist or protocols for the healthcare professionals to record their verification. In some instances, the suggestions comprising access or reminder to secure and review relevant documentations prior to the procedure. Examples of relevant documentations include medical history, lab test results, consent form, pre-anesthesia assessment, reports of radiology, pathology, or biopsy, and blood work.

In some embodiments, the systems, methods, software, and platforms as described herein provide healthcare professionals to perform the following steps prior to approving the individual for receiving the procedure: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians.

In some embodiments, the suggestions of pre-procedure phase instruct the healthcare professionals to confirm or mark the site of procedure. In some cases, if the suggestions of the pre-procedure phase are not adequality followed, recorded, or tracked, additional suggestion in a form a warning can alarm the healthcare professionals who are performing the procedure. In some embodiments, the healthcare professionals can confirm, deny, modify, add, remove, shift, analyze, override, suspend, record, monitor, and track the suggestions of pre-procedure phase. In some embodiments, suggestions of pre-procedure phase can not be changed. In some embodiments, suggestions of pre-procedure phase must be followed by healthcare professionals. In some embodiments, certain healthcare professionals can examine and overrule suggestions of other healthcare professionals. In some embodiments, healthcare professionals can examine and verify whether other healthcare professionals are adhering to the suggestions. In some instances, suggestions of pre-procedure phase and identifier generated and modified during pre-procedure phase can at least partially determine suggestions of any other phases. For example, healthcare professionals can suspend or change suggestion for procedure phase based on test results of diagnosis phase or based on observations made during pre-procedure phase. In some embodiments, suggestions of pre-procedure phase are first suggestions and can at least partially determine suggestions of other phases. In some embodiments, suggestions of pre-procedure phases are the second suggestions. In some cases, suggestions of pre-procedure phase can be both first and second suggestions. In some embodiments, suggestions for pre-procedure can be first suggestions and provided to a first user for treating and caring the individual during a first phase of pre-procedure. In some cases, the first user is the individual. In some cases, the first user is a healthcare professional. In some alternatives, the first user is not a healthcare professional. In some cases, suggestions for pre-procedure are second suggestions and provided to a second user for treating and caring the individual during a second phase of pre-procedure. In some instances, the second user is a healthcare professional.

### Procedure

In some embodiments, the systems, methods, software, and platforms as described herein provide suggestions for healthcare professional to perform a procedure to the individual. Procedure can include an act, method, or manner of proceeding in some action especially the sequence of the steps to be followed. In some cases, the procedures can comprise a very accurate, precisely targeted sequence of steps. In some embodiments, procedure comprises the sequence of steps to be followed in establishing course of action. In some cases, procedure comprises diagnosis described previously. In some cases, suggestions for procedure are based on identifier of the individual. In some instances, the identifier has been generated or modified by any other phases of the plurality of phases of medical care encounter. In some embodiments, suggestions for procedure phase can be at least partially determined by suggestions of any other phase of the plurality of phases. For example, suggestions for procedure can be modified, suspended, or overridden based on results of diagnosis or lab tests. In some embodiments, the procedure can be administering of a therapy such as thrombosis prophylaxis, precordial thump, politzerization, hemodialysis, hemofiltration, plasmapheresis, apheresis, extracorporeal membrane oxygenation, cancer immunotherapy, cancer vaccine, cervical conization, chemotherapy, cytoluminescent therapy, insulin potentiation therapy, low-dose chemotherapy, monoclonal antibody therapy, photodynamic therapy, radiation therapy, targeted therapy, tracheal intubation, unsealed source radiotherapy. virtual reality therapy, physical therapy, physiotherapy, speech therapy, phototherapy, hydrotherapy, heat therapy, shock therapy, insulin shock therapy, electroconvulsive therapy, symptomatic treatment, fluid replacement therapy, palliative care, hyperbaric oxygen therapy, oxygen therapy, gene therapy, enzyme replacement therapy, intravenous therapy, phage therapy, respiratory therapy, vision therapy, electrotherapy, transcutaneous electrical nerve stimulation, laser therapy, combination therapy, occupational therapy, immunization, vaccination, immunosuppressive therapy, psychotherapy, drug therapy, acupuncture, antivenom, magnetic therapy, craniosacral therapy, chelation therapy, hormonal therapy, hormone replacement therapy, opiate replacement therapy, cell therapy, stem cell treatments, intubation, nebulization, inhalation therapy. particle therapy. proton therapy, fluoride therapy, cold compression therapy, animal-assisted therapy, negative pressure wound therapy, nicotine replacement therapy, or oral rehydration therapy. Exemplary procedures can include: acute management of malignant hyperthermia, administering blood product, arterial blood gas collection, arterial pressure monitor, assisted manual ventilation, chest tube insertion thoracostomy, chest tube management, chest tube removal, continuous enteral feeding, endotracheal suctioning, external ventricular drain care, extubation after prolonged mechanical ventilation, fluid resuscitation, intracranial pressure monitoring, initial increased intracranial pressure therapy, initiate mechanical ventilation, mechanical ventilation management, nasogastric tube insertion, needle decompression, orogastric tube insertion, PICC central line insertion, pain assessment, patient controlled analgesia, peritoneal catheter management, peritoneal catheter removal, pressure control mode, progressive therapy for increased intracranial pressure, radial artery cannulation, sedation and pain management for procedures, skin traction application, urinary catheter insertion for female, urinary catheter insertion for male, vacuum-assisted closure therapy dressing change, vacuum-assisted closure therapy, volume control mode, weaning from prolonged mechanical ventilation using pressure support, cervical collar application, cervical collar skin care, intraosseous needle insertion, pain management, rapid sequence intubation, saphenous vein cutdown, sedation and pain management, taping the endotracheal tube, or tourniquet application.

In some embodiments, the procedure comprises surgery. In some embodiments, the surgery can include a branch of medicine dealing with working with the hands, handicraft, or skill for treatment of disease, injury, or deformity by manual or instrumental procedures. In some cases, the surgery can be elective, semi-elective, emergency, cosmetic, exploratory, amputation, replantation, reconstructive, transplant, surgery of body parts, minimally invasive, laparoscopic, angioplasty, open surgical procedure, laser surgery, robotic surgery, or microsurgery. In some cases, the suggestions instruct and provide protocols for the healthcare professions on performing surgery such as decompressive craniectomy, hemispherectomy, anterior temporal lobectomy, hypophysectomy, amygdalohippocampectomy, ventriculostomy, craniotomy, pallidotomy, thalamotomy, lobotomy, bilateral cingulotomy, cordotomy, rhizotomy, neurosurgery, psychosurgery, brain biopsy, peripheral nervous system, ganglionectomy, sympathectomy, endoscopic thoracic sympathectomy, neurectomy, axotomy, vagotomy, nerve biopsy, hypophysectomy, thyroidectomy, parathyroidectomy, adrenalectomy, pinealectomy, punctoplasty, trabeculoplasty, photorefractive keratectomy, trabeculectomy, iridectomy, vitrectomy, dacryocystorhinostomy, radial keratotomy, mini asymmetric radial keratotomy, corneal transplantation, otoplasty, stapedectomy, mastoidectomy, auriculectomy, myringotomy, rhinoplasty, septoplasty, hinectomy, laryngectomy, pneumonectomy, racheostomy, sinusotomy, pneumotomy, cricothyroidotomy, cricothyrotomy, bronchotomy, thoracotomy, thyrotomy, tracheotomy, lateral rhinotomy, pleurodesis, lung transplantation, angioplasty, valvuloplasty, pericardiectomy, endarterectomy, cardiotomy, pericardiotomy, heart transplantation, tonsillectomy, adenoidectomy, thymectomy, splenectomy, lymphadenectomy, thymus transplantation, spleen transplantation, splenopexy, lymph node biopsy, uvulopalatoplasty, palatoplasty, gingivectomy, glossectomy, esophagectomy, gastrectomy, appendectomy, proctocolectomy, colectomy, hepatectomy, cholecystectomy, pancreatectomy, pancreaticoduodenectomy, gastrostomy, percutaneous endoscopic gastrostomy, gastroduodenostomy, gastroenterostomy, ileostomy, jejunostomy, colostomy, cholecystostomy, hepatoportoenterostomy, sigmoidostomv, uvlotomy, myotomy, Heller myotomy, pyloromyotomy, anal sphincterotomy, lateral internal sphincterotomy, vertical banded gastroplasty, gastropexy, colon resection, Nissen fundoplication, hernia repair, pmentopexy, liver biopsy, urethroplasty, pyeloplasty, nephrectomy, cystectomy, nephrostomy, ureterostomy, cystostomy (Suprapubic cystostomy), urostomy, nephrotomy, nephropexy, urethropexy, lithotripsy, kidney transplantation, renal biopsy, phalloplasty, scrotoplasty, vasectomy, penectomy, orchidectomy, prostatectomy, posthectomy, gonadectomy, vasovasostomy, vasoepididymostomy, meatotomy, circumcision, foreskin restoration, orchiopexy, prostate biopsy, vaginoplasty, clitoroplasty, labiaplasty, tuboplasty, fimbrioplasty, cervicectomy, clitoridectomy, oophorectomy, salpingoophorectomy, salpingectomy, hysterectomy, vaginectomy, vulvectomy, salpingostomy, amniotomy, clitoridotomy, hysterotomy, hymenotomy, episiotomy, symphysiotomy, tubal ligation, tubal reversal, colporrhaphy, cesarean section, hymenorrhaphy, endometrial biopsy, acromioplasty, khyphoplasty, mentoplastym, acromioplasty, arthroplasty, rotationplasty, ostectomy, femoral head ostectomy, vertebrectomy, coccygectomy, astragalectomy, corpectomy, facetectomy, laminectomy, hemilaminectomy, synovectomy, discectomy, osteotomy, arthrotomy, laminotomy, foraminotomy, epiphysiodesis, arthrodesis, arthroscopy, ulnar collateral ligament reconstruction, bursectomy, amputation, hemicorporectomy, hemipelvectomy, myotomy, tenotomy, fasciotomy, muscle biopsy, amputation, tendon transfer, mammoplasty, lumpectomy, mastectomy, breast implant, mastopexy, breast reconstruction, breast reduction plasty, V-plasty, VY-plasty, W-plasty, Z-plasty, scharotomy, skin biopsy, abdominoplasty, hernioplasty, frenuloplasty, Z-plasty, diverticulectomy, frenectomy, hemorrhoidectomy. Mastoidectomy, thrombectomy, embolectomy, ganglionectomy, lobectomy, myomectomy, panniculectomy, reterosigmoidostomy, fistulotomy, laparotomy, myringotomy, sphincterotomy, commissurotomy, abdominal surgery, onguinal hernia surgery, biopsy, brostrom procedure, cauterization, grafting, hypnosurgery, laparoscopy, Nuss procedure, or radiosurgery.

In some cases, the systems, methods, software, and platforms as described herein provide suggestions such as instructions and protocols for healthcare professionals during before, during, and after procedure to: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians.

In some embodiments, the systems, methods, software, and platforms disclosed herein allow healthcare professionals to confirm, deny, modify, add, remove, shift, analyze, override, suspend, record, monitor, and track the suggestions of procedure phase. In some embodiments, suggestions of procedure phase can not be changed. In some embodiments, certain healthcare professionals can examine and overrule suggestions of other healthcare professionals. For instances, physician can suspend or cancel suggestions of procedure based on test results from clinical or pathology lab. In some embodiments, healthcare professionals can examine and verify whether other healthcare professionals are adhering to the suggestions. In some embodiments, suggestions of procedure phase must be followed by healthcare professionals. In some embodiments, suggestions of the procedure phase and identifier generated and modified by procedure phase can at least partially determine suggestions of any other phases. In some embodiments, suggestions of procedure phase are first suggestions and can at least partially determine second suggestions of other phases. In some embodiments, suggestions of procedure phase are second suggestions. In some cases, suggestions of procedure phase can be both first and second suggestions. In some embodiments, suggestions for procedure are first suggestions and provided to a first user for treating and caring the individual during a first phase of procedure. In some cases, the first user is the individual. In some cases, the first user is a healthcare professional. In some alternatives, the first user is not a healthcare professional. In some cases, suggestions for procedure are second suggestions and provided to a second user for treating and caring the individual during a second phase of procedure. In some instances, the second user is a healthcare professional.

### Post-Procedure (Post-Op)

In some embodiments, the systems, methods, software, and platforms as described herein provide suggestions for healthcare professional to perform post-procedure care during post-procedure phase of the plurality of phases of medical care encounter experienced by the individual. In some alternatives, suggestions for post-procedure can be based on identifier of the individual. In some cases, the identifier has been generated or modified by any other phases of the plurality of phases of medical care encounter. In some embodiments, suggestions for post-procedure phase can be at least partially determined by suggestions of any other phase of the plurality of phases. In some instances, suggestions can be modified by healthcare professionals such as postoperative nurses. In some cases, suggestions can be modified based on physician's input. In some cases, suggestions can be modified based on results of lab testing. In some instances, suggestions can be examined or overridden. In some embodiments, suggestions of post-procedure comprise monitoring and recording the individual's vital signs, pain management, rate and type of intravenous fluid, urine and gastrointestinal fluid out, medications, laboratory testing and results, comments from the individual and healthcare professionals, observation and assessment made by both the individual and the healthcare professionals, complications, analgesia, breathing, mobility, responsiveness, or any other any other changes regarding the status of the individual and medical care encounters experienced by the individual. In some cases, the post-procedure suggestions can notify both the individual and the healthcare professionals to ensure additional cares such as early mobilization, adequate nutrition, adequate pain control, and prevention of skin breakdown are received by the individual. In some instances, the suggestions can instruct discharging of the individual by healthcare professionals diagnosing the individual for readiness to be discharged, providing summary of the entire medical care encounter, and instruction for further management, including medicine prescribed and follow-up appointments.

In some cases, post-procedure suggestions instruct monitoring the individual for signs of restlessness such as airway obstruction, hypoxia, both internal external hemorrhage, hypotension, hypertension, postoperative pain, shivering, hypothermia, vomiting, aspiration, falling on the floor, or residual narcosis. In some embodiments, the healthcare professionals can confirm, deny, modify, add, remove, shift, analyze, override, suspend, record, monitor, and track the suggestions of post-procedure phase. In some embodiments, suggestions of post-procedure phase can not be changed. In some embodiments, suggestions of post-procedure phase must be followed by healthcare professionals. In some cases, certain healthcare professionals can examine and overrule suggestions of other healthcare professionals. In some embodiments, healthcare professionals can examine and verify whether other healthcare professionals are adhering to the suggestions. For example, physician can examine whether the attending nurses are checking the individual based on schedule as determined by the suggestions. Alternatively, physician can modify suggestion of discharging the individual based on recovery progress of the individual.

In some embodiments, the systems, methods, software, and platforms as described herein provide suggestions such as instructions and protocols for healthcare professionals to: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians. In some instances, suggestions of post-procedure phase and the identifier generated and modified during post-procedure phase can at least partially determine suggestions of any other phases. In some embodiments, suggestions of post-procedure phase are first suggestions and can at least partially determine second suggestions of any of the plurality of phases. In some embodiments, suggestions of post-procedure phases are second suggestions. In some cases, suggestions of post-procedure phase can be both first and second suggestions. In some instances, suggestions for post-procedure are first suggestions and provided to a first user for treating and caring the individual during a first phase of post-procedure. In some cases, the first user is the individual. In some cases, the first user is a healthcare professional. In some cases, the first user is not a healthcare professional. In some embodiments, suggestions for post-procedure are second suggestions and provided to a second user for treating and caring the individual during a second phase of post-procedure. In some embodiments, the second user is a healthcare professional.

### Pre-Operation

In some embodiments, the systems, methods, software, and platforms as described herein provide suggestions for healthcare professionals to care for the individual prior to performing an operation to the individual. In some alternatives, suggestions for the pre-operation are based on identifier of the individual. In some cases, identifier has been generated or modified by any other phases of the plurality of phases of medical care encounter. In some embodiments, suggestions for pre-operation phase can be at least partially determined by suggestions of any other phase of the plurality of phases. In some instances, the suggestions instruct verifying the operation with both the individual and the healthcare professionals. In some instances, the verification comprises correct identity of the individual, correct operation type, correct site of operation, and correct items for the operation. In some embodiments, the suggestions comprise a checklist or protocols for the healthcare professionals to record their verification. In some instances, the suggestions comprising access or reminder to secure and review relevant documentations prior to the operation. Examples of relevant documentations include medical history, lab test results, consent form, pre-anesthesia assessment, reports of radiology, pathology, or biopsy, and blood work.

In some embodiments, the systems, methods, software, and platforms as described herein provide healthcare professionals to perform the following steps prior to approving the individual for receiving the operation: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians.

In some embodiments, the suggestions of pre-operation phase instruct the healthcare professionals to confirm or mark the site of operation. In some cases, if the suggestions of the pre-operation phase are not adequality followed, recorded, or tracked, additional suggestion in a form a warning can alarm the healthcare professionals who are performing the operation. In some embodiments, the healthcare professionals can confirm, deny, modify, add, remove, shift, analyze, override, suspend, record, monitor, and track the suggestions of pre-operation phase. In some embodiments, suggestions of pre-operation phase cannot be changed. In some embodiments, suggestions of pre-operation phase must be followed by healthcare professionals. In some embodiments, certain healthcare professionals can examine and overrule suggestions of other healthcare professionals. In some embodiments, healthcare professionals can examine and verify whether other healthcare professionals are adhering to the suggestions. In some instances, suggestions of pre-operation phase and identifier generated and modified during pre-operation phase can at least partially determine suggestions of any other phases. For example, healthcare professionals can suspend or change suggestion for operation phase based on test results of diagnosis phase or based on observations made during pre-operation phase. In some embodiments, suggestions of pre-operation phase are first suggestions and can at least partially determine suggestions of other phases. In some embodiments, suggestions of pre-operation phases are the second suggestions. In some cases, suggestions of pre-operation phase can be both first and second suggestions. In some embodiments, suggestions for pre-operation can be first suggestions and provided to a first user for treating and caring the individual during a first phase of pre-operation. In some cases, the first user is the individual. In some cases, the first user is a healthcare professional. In some alternatives, the first user is not a healthcare professional. In some cases, suggestions for pre-operation are second suggestions and provided to a second user for treating and caring the individual during a second phase of pre-operation. In some instances, the second user is a healthcare professional.

### Operation

In some embodiments, the systems, methods, software, and platforms as described herein provide suggestions for healthcare professional to perform an operation to the individual. In some embodiments, the operation can include any movement or series of movements made in carrying out a strategic plan. In some embodiments, the operation can include a process or action that is part of a series in any specific plan, project, or venture. In some cases, operation comprises diagnosis described previously. In some cases, suggestions for operation are based on identifier of the individual. In some instances, the identifier has been generated or modified by any other phases of the plurality of phases of medical care encounter. In some embodiments, suggestions for operation phase can be at least partially determined by suggestions of any other phase of the plurality of phases. For example, suggestions for operation can be modified, suspended, or overridden based on results of diagnosis or lab tests. In some embodiments, the operation can be administering of a therapy such as thrombosis prophylaxis, precordial thump, politzerization, hemodialysis, hemofiltration, plasmapheresis, apheresis, extracorporeal membrane oxygenation, cancer immunotherapy, cancer vaccine, cervical conization, chemotherapy, cytoluminescent therapy, insulin potentiation therapy, low-dose chemotherapy, monoclonal antibody therapy, photodynamic therapy, radiation therapy, targeted therapy, tracheal intubation, unsealed source radiotherapy. virtual reality therapy, physical therapy, physiotherapy, speech therapy, phototherapy, hydrotherapy, heat therapy, shock therapy, insulin shock therapy, electroconvulsive therapy, symptomatic treatment, fluid replacement therapy, palliative care, hyperbaric oxygen therapy, oxygen therapy, gene therapy, enzyme replacement therapy, intravenous therapy, phage therapy, respiratory therapy, vision therapy, electrotherapy, transcutaneous electrical nerve stimulation, laser therapy, combination therapy, occupational therapy, immunization, vaccination, immunosuppressive therapy, psychotherapy, drug therapy, acupuncture, antivenom, magnetic therapy, craniosacral therapy, chelation therapy, hormonal therapy, hormone replacement therapy, opiate replacement therapy, cell therapy, stem cell treatments, intubation, nebulization, inhalation therapy. particle therapy. proton therapy, fluoride therapy, cold compression therapy, animal-assisted therapy, negative pressure wound therapy, nicotine replacement therapy, or oral rehydration therapy. Exemplary operation can include thoracostomy, mini-laparotomy to insert peritoneal tube, initial look damage control laparotomy, second look damage control laparotomy, craniotomy for epidural hematoma, craniotomy for subdural hematoma, craniectomy, intraparenchymal bolt insertion, ventriculostomy, spica cast, pavlik harness, elastic intramedullary nails, external fixation for femur fracture, antegrade rigid intramedullary nail, open femur fracture with external fixation, open femur fracture—undesignated fracture reduction, skin graft, or two incision four compartment fasciotomy—lower extremity.

In some embodiments, the operation comprises surgery. In some embodiments, the surgery can include a branch of medicine dealing with working with the hands, handicraft, or skill for treatment of disease, injury, or deformity by manual or instrumental operations. In some cases, the surgery can be elective, semi-elective, emergency, cosmetic, exploratory, amputation, replantation, reconstructive, transplant, surgery of body parts, minimally invasive, laparoscopic, angioplasty, open surgical operation, laser surgery, robotic surgery, or microsurgery. In some cases, the suggestions instruct and provide protocols for the healthcare professions on performing surgery such as decompressive craniectomy, hemispherectomy, anterior temporal lobectomy, hypophysectomy, amygdalohippocampectomy, ventriculostomy, craniotomy, pallidotomy, thalamotomy, lobotomy, bilateral cingulotomy, cordotomy, rhizotomy, neurosurgery, psychosurgery, brain biopsy, peripheral nervous system, ganglionectomy, sympathectomy, endoscopic thoracic sympathectomy, neurectomy, axotomy, vagotomy, nerve biopsy, hypophysectomy, thyroidectomy, parathyroidectomy, adrenalectomy, pinealectomy, punctoplasty, trabeculoplasty, photorefractive keratectomy, trabeculectomy, iridectomy, vitrectomy, dacryocystorhinostomy, radial keratotomy, mini asymmetric radial keratotomy, corneal transplantation, otoplasty, stapedectomy, mastoidectomy, auriculectomy, myringotomy, rhinoplasty, septoplasty, hinectomy, laryngectomy, pneumonectomy, racheostomy, sinusotomy, pneumotomy, cricothyroidotomy, cricothyrotomy, bronchotomy, thoracotomy, thyrotomy, tracheotomy, lateral rhinotomy, pleurodesis, lung transplantation, angioplasty, valvuloplasty, pericardiectomy, endarterectomy, cardiotomy, pericardiotomy, heart transplantation, tonsillectomy, adenoidectomy, thymectomy, splenectomy, lymphadenectomy, thymus transplantation, spleen transplantation, splenopexy, lymph node biopsy, uvulopalatoplasty, palatoplasty, gingivectomy, glossectomy, esophagectomy, gastrectomy, appendectomy, proctocolectomy, colectomy, hepatectomy, cholecystectomy, pancreatectomy, pancreaticoduodenectomy, gastrostomy, percutaneous endoscopic gastrostomy, gastroduodenostomy, gastroenterostomy, ileostomy, jejunostomy, colostomy, cholecystostomy, hepatoportoenterostomy, sigmoidostomv, uvlotomy, myotomy, Heller myotomy, pyloromyotomy, anal sphincterotomy, lateral internal sphincterotomy, vertical banded gastroplasty, gastropexy, colon resection, Nissen fundoplication, hernia repair, pmentopexy, liver biopsy, urethroplasty, pyeloplasty, nephrectomy, cystectomy, nephrostomy, ureterostomy, cystostomy (Suprapubic cystostomy), urostomy, nephrotomy, nephropexy, urethropexy, lithotripsy, kidney transplantation, renal biopsy, phalloplasty, scrotoplasty, vasectomy, penectomy, orchidectomy, prostatectomy, posthectomy, gonadectomy, vasovasostomy, vasoepididymostomy, meatotomy, circumcision, foreskin restoration, orchiopexy, prostate biopsy, vaginoplasty, clitoroplasty, labiaplasty, tuboplasty, fimbrioplasty, cervicectomy, clitoridectomy, oophorectomy, salpingoophorectomy, salpingectomy, hysterectomy, vaginectomy, vulvectomy, salpingostomy, amniotomy, clitoridotomy, hysterotomy, hymenotomy, episiotomy, symphysiotomy, tubal ligation, tubal reversal, colporrhaphy, cesarean section, hymenorrhaphy, endometrial biopsy, acromioplasty, khyphoplasty, mentoplastym, acromioplasty, arthroplasty, rotationplasty, ostectomy, femoral head ostectomy, vertebrectomy, coccygectomy, astragalectomy, corpectomy, facetectomy, laminectomy, hemilaminectomy, synovectomy, discectomy, osteotomy, arthrotomy, laminotomy, foraminotomy, epiphysiodesis, arthrodesis, arthroscopy, ulnar collateral ligament reconstruction, bursectomy, amputation, hemicorporectomy, hemipelvectomy, myotomy, tenotomy, fasciotomy, muscle biopsy, amputation, tendon transfer, mammoplasty, lumpectomy, mastectomy, breast implant, mastopexy, breast reconstruction, breast reduction plasty, V-plasty, VY-plasty, W-plasty, Z-plasty, scharotomy, skin biopsy, abdominoplasty, hernioplasty, frenuloplasty, Z-plasty, diverticulectomy, frenectomy, hemorrhoidectomy. Mastoidectomy, thrombectomy, embolectomy, ganglionectomy, lobectomy, myomectomy, panniculectomy, reterosigmoidostomy, fistulotomy, laparotomy, myringotomy, sphincterotomy, commissurotomy, abdominal surgery, onguinal hernia surgery, biopsy, brostrom operation, cauterization, grafting, hypnosurgery, laparoscopy, Nuss operation, or radiosurgery.

In some cases, the systems, methods, software, and platforms as described herein provide suggestions such as instructions and protocols for healthcare professionals during before, during, and after operation to: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians. In some embodiments, the systems, methods, software, and platforms disclosed herein allow healthcare professionals to confirm, deny, modify, add, remove, shift, analyze, override, suspend, record, monitor, and track the suggestions of operation phase. In some embodiments, suggestions of operation phase can not be changed. In some embodiments, certain healthcare professionals can examine and overrule suggestions of other healthcare professionals. For instances, physician can suspend or cancel suggestions of operation based on test results from clinical or pathology lab. In some embodiments, healthcare professionals can examine and verify whether other healthcare professionals are adhering to the suggestions. In some embodiments, suggestions of operation phase must be followed by healthcare professionals. In some embodiments, suggestions of the operation phase and identifier generated and modified by operation phase can at least partially determine suggestions of any other phases. In some embodiments, suggestions of operation phase are first suggestions and can at least partially determine second suggestions of other phases. In some embodiments, suggestions of operation phase are second suggestions. In some cases, suggestions of operation phase can be both first and second suggestions. In some embodiments, suggestions for operation are first suggestions and provided to a first user for treating and caring the individual during a first phase of operation. In some cases, the first user is the individual. In some cases, the first user is a healthcare professional. In some alternatives, the first user is not a healthcare professional. In some cases, suggestions for operation are second suggestions and provided to a second user for treating and caring the individual during a second phase of operation. In some instances, the second user is a healthcare professional.

### Post-Operation (Post-Op)

In some embodiments, the systems, methods, software, and platforms as described herein provide suggestions for healthcare professional to perform post-operation care during post-operation phase of the plurality of phases of medical care encounter experienced by the individual. In some alternatives, suggestions for post-operation can be based on identifier of the individual. In some cases, the identifier has been generated or modified by any other phases of the plurality of phases of medical care encounter. In some embodiments, suggestions for post-operation phase can be at least partially determined by suggestions of any other phase of the plurality of phases. In some instances, suggestions can be modified by healthcare professionals such as postoperative nurses. In some cases, suggestions can be modified based on physician's input. In some cases, suggestions can be modified based on results of lab testing. In some instances, suggestions can be examined or overridden. In some embodiments, suggestions of post-operation comprise monitoring and recording the individual's vital signs, pain management, rate and type of intravenous fluid, urine and gastrointestinal fluid out, medications, laboratory testing and results, comments from the individual and healthcare professionals, observation and assessment made by both the individual and the healthcare professionals, complications, analgesia, breathing, mobility, responsiveness, or any other any other changes regarding the status of the individual and medical care encounters experienced by the individual. In some cases, the post-operation suggestions can notify both the individual and the healthcare professionals to ensure additional cares such as early mobilization, adequate nutrition, adequate pain control, and prevention of skin breakdown are received by the individual. In some instances, the suggestions can instruct discharging of the individual by healthcare professionals diagnosing the individual for readiness to be discharged, providing summary of the entire medical care encounter, and instruction for further management, including medicine prescribed and follow-up appointments.

In some cases, post-operation suggestions instruct monitoring the individual for signs of restlessness such as airway obstruction, hypoxia, both internal external hemorrhage, hypotension, hypertension, postoperative pain, shivering, hypothermia, vomiting, aspiration, falling on the floor, or residual narcosis. In some embodiments, the healthcare professionals can confirm, deny, modify, add, remove, shift, analyze, override, suspend, record, monitor, and track the suggestions of post-operation phase. In some embodiments, suggestions of post-operation phase can not be changed. In some embodiments, suggestions of post-operation phase must be followed by healthcare professionals. In some cases, certain healthcare professionals can examine and overrule suggestions of other healthcare professionals. In some embodiments, healthcare professionals can examine and verify whether other healthcare professionals are adhering to the suggestions. For example, physician can examine whether the attending nurses are checking the individual based on schedule as determined by the suggestions. Alternatively, physician can modify suggestion of discharging the individual based on recovery progress of the individual.

In some embodiments, the systems, methods, software, and platforms as described herein provide suggestions such as instructions and protocols for healthcare professionals to: confirm identifier or profile associated with the individual; review communications from previous healthcare professionals who provide previous care; perform diagnosis; look for signs of hypothermia, acidosis, and coagulopathy; ensure airway, breathing, and circulation of the individual; perform neurovascular assessment; perform sedation or pain management; communicate with family or legal guardians. In some instances, suggestions of post-operation phase and the identifier generated and modified during post-operation phase can at least partially determine suggestions of any other phases. In some embodiments, suggestions of post-operation phase are first suggestions and can at least partially determine second suggestions of any of the plurality of phases. In some embodiments, suggestions of post-operation phases are second suggestions. In some cases, suggestions of post-operation phase can be both first and second suggestions. In some instances, suggestions for post-operation are first suggestions and provided to a first user for treating and caring the individual during a first phase of post-operation. In some cases, the first user is the individual. In some cases, the first user is a healthcare professional. In some cases, the first user is not a healthcare professional. In some embodiments, suggestions for post-operation are second suggestions and provided to a second user for treating and caring the individual during a second phase of post-operation. In some embodiments, the second user is a healthcare professional.

### Critical Care

In some embodiments, the systems, methods, software, and platforms as described herein provide suggestions for healthcare professionals to care for the individual during a critical care phase of the medical care encounter. In some embodiments, the suggestions can be for critical care performed by the healthcare professionals at a point-of-care capacity. In some embodiments, suggestions for the critical care phase are based on identifier of the individual. In some cases, identifier has been generated or modified by any other phases of the plurality of phases of medical care encounter. In some embodiments, suggestions for critical care phase can be at least partially determined by suggestions of any other phase of the plurality of phases. In some embodiments, suggestions for critical care phase can at least partially determine suggestions of any other phase of the plurality of phases.

In some instances, the provided suggestions for critical care phase can include treating the individual with severe or life-threatening illness or injury. The treatment can include constant care and supervision from healthcare professionals as part of the neonatal intensive care unit, pediatric intensive care unit, psychiatric intensive care unit, coronary care unit, neurological intensive care unit, intensive care unit, post-anesthesia unit, high dependency unit, surgical intensive care unit, or mobile intensive unit. Non-limiting examples of illness or injury that can be treated include sepsis, traumatic brain injury, shock, stroke, aneurysm, trauma, post-operative intensive care, cancer-related intensive care, heart failure, pneumonia, and respiratory failure. Suggestions can include protocols for treating the individual and protocols on using medical devices such as a ventilator, monitoring equipment, IV lines and pumps, feeding tubes, drains, and catheters. Other exemplary suggestions of critical care can include instructions and protocols for healthcare professionals to: confirm patient identifier or profile; review communications from previous healthcare professionals who provide previous care; perform diagnosis; look for signs of hypothermia, acidosis, and coagulopathy; ensure airway, breathing, and circulation of the individual; perform assessment; and perform sedation or pain management.

In some embodiments, the suggestions of critical phase instruct the healthcare professionals to inform the family or legal guardians of the individual. In some embodiments, if the suggestions are not adequality followed, recorded, or tracked, additional suggestion in a form of a warning can alert the healthcare professionals. In some embodiments, the healthcare professionals can confirm, deny, modify, add, remove, shift, analyze, override, suspend, record, monitor, and track the suggestions of critical care phase. In some embodiments, suggestions of critical care phase can not be changed. In some embodiments, suggestions of critical care phase must be followed by healthcare professionals. In some embodiments, certain healthcare professionals can examine and overrule suggestions of other healthcare professionals. In some embodiments, healthcare professionals can examine and verify whether other healthcare professionals are adhering the suggestions. In some embodiments, suggestions of critical care phase and identifier generated and modified during critical care phase can at least partially determine suggestions of any other phases. For example, healthcare professionals can suspend or change suggestion for procedure or operation phase based on results or observations made during critical care phase. In some embodiments, suggestions of critical phase are first suggestions and can at least partially determine suggestions of other phases. In some embodiments, suggestions of critical care phase are the second suggestions. In some cases, suggestions of critical care phase can be both first and second suggestions. In some embodiments, suggestions for critical care can be first suggestions and provided to a first user for treating and caring the individual during a first phase of critical care. In some cases, the first user is the individual. In some cases, the first user is a healthcare professional. In some alternatives, the first user is not a healthcare professional. In some cases, suggestions for critical care are second suggestions and provided to a second user for treating and caring the individual during a second phase of critical care. In some instances, the second user is a healthcare professional.

### After Care

In some instances, the systems, methods, software, and platforms as described herein provide suggestions for healthcare professionals to care for the individual during an after care phase of the medical care encounter. In some embodiments, the suggestions can be for after care performed by the healthcare professionals just prior to discharging of the individual from the medical care facility. In some embodiments, suggestions for the after care phase are based on identifier of the individual. In some cases, identifier has been generated or modified by any other phases of the plurality of phases of medical care encounter. In some embodiments, suggestions for after care phase can be at least partially determined by suggestions of any other phase of the plurality of phases. In some embodiments, suggestions for after care phase can at least partially determine suggestions of any other phase of the plurality of phases.

In some instances, the provided suggestions can include instructions and protocols for healthcare professionals to: confirm patient identifier or profile; review communications from previous healthcare professionals who provide previous care; perform diagnosis; look for signs of hypothermia, acidosis, coagulopathy, and infection; ensure airway, breathing, and circulation of the individual; perform assessment; perform sedation or pain management; position the individual for comfort; care for skin traction; provide physical therapy; and review criteria for discharging the individual.

In some embodiments, the suggestions of after care phase instruct the healthcare professionals to inform the family or legal guardians of the individual. In some embodiments, if the suggestions are not adequality followed, recorded, or tracked, additional suggestion in a form a warning can alert the healthcare professionals. In some embodiments, the healthcare professionals can confirm, deny, modify, add, remove, shift, analyze, override, suspend, record, monitor, and track the suggestions of after care phase. In some embodiments, suggestions of after care phase can not be changed. In some embodiments, suggestions of after care phase must be followed by healthcare professionals. In some embodiments, certain healthcare professionals can examine and overrule suggestions of other healthcare professionals. In some embodiments, healthcare professionals can examine and verify whether other healthcare professionals are adhering the suggestions. In some embodiments, suggestions of after care phase and identifier generated and modified during after care phase can at least partially determine suggestions of any other phases. For example, healthcare professionals can suspend or change suggestion for discharge phase based on results or observations made during after care phase. In some instances, suggestions of after phase are first suggestions and can at least partially determine suggestions of other phases. In some embodiments, suggestions of after care phase are the second suggestions. In some cases, suggestions of after care phase can be both first and second suggestions. In some embodiments, suggestions for after care can be first suggestions and provided to a first user for treating and caring the individual during a first phase of after care. In some cases, the first user is the individual. In some cases, the first user is a healthcare professional. In some alternatives, the first user is not a healthcare professional. In some cases, suggestions for after care are second suggestions and provided to a second user for treating and caring the individual during a second phase of after care. In some instances, the second user is a healthcare professional.

### Discharge

In some instances, the systems, methods, software, and platforms as described herein provide suggestions for healthcare professionals to care and prepare the individual for a discharge phase of the medical care encounter. In some embodiments, suggestions for the discharge phase are based on identifier of the individual. In some cases, identifier has been generated or modified by any other phases of the plurality of phases of medical care encounter. In some embodiments, suggestions for discharge phase can be at least partially determined by suggestions of any other phase of the plurality of phases. In some embodiments, suggestions for discharge phase can at least partially determine suggestions of any other phases of the plurality of phases in a future medical encounter experienced by the individual.

In some instances, the provided suggestions can include instructions and protocols for healthcare professionals to: confirm patient identifier or profile; look for signs of hypothermia, acidosis; restore regular diet for the individual; schedule follow-up appointment; and manage pain and prescriptions. In some cases, if the individual is a pediatric patient the suggestion for discharge phase can include recommendations on how to prepare for a return to school. In some cases, the suggestions alert the healthcare professionals to follow up with the individual after the individual has already been discharged from a medical care facility.

In some embodiments, the suggestions of discharge phase instruct the healthcare professionals to inform the family or legal guardians of the individual. In some cases, the suggestions can include protocols, checklists, and instructions for both healthcare professionals and family or legal guardians of the individual to properly care for the individual in a non-medical care facility setting. In some embodiments, the suggestions can include protocols, checklists, and instructions for the individual or the family or legal guardians of the individual to properly care for the individual in a non-medical care facility setting. In some embodiments, the suggestions can include protocols, checklists, and instructions for the individual or the family or legal guardians of the individual to properly care for the individual in a home setting.

In some embodiments, if the suggestions are not adequality followed, recorded, or tracked, additional suggestion in a form a warning can alert the healthcare professionals. In some embodiments, the healthcare professionals can confirm, deny, modify, add, remove, shift, analyze, override, suspend, record, monitor, and track the suggestions of discharge phase. In some embodiments, suggestions of discharge phase can not be changed. In some embodiments, suggestions of discharge phase must be followed by healthcare professionals. In some embodiments, certain healthcare professionals can examine and overrule suggestions of other healthcare professionals. In some embodiments, healthcare professionals can examine and verify whether other healthcare professionals are adhering the suggestions. In some embodiments, suggestions of discharge phase and identifier generated and modified during discharge phase can at least partially determine suggestions of any other phases. For example, healthcare professionals can suspend or change suggestion for discharge phase based on results or observations made during after care phase. In some instances, suggestions of discharge phase are first suggestions and can at least partially determine suggestions of other phases. In some embodiments, suggestions of discharge phase are the second suggestions. In some cases, suggestions of discharge phase can be both first and second suggestions. In some embodiments, suggestions for discharge can be first suggestions and provided to a first user for treating and caring the individual during a first phase of discharge. In some cases, the first user is the individual. In some cases, the first user is a healthcare professional. In some alternatives, the first user is not a healthcare professional. In some cases, suggestions for discharge phase are second suggestions and provided to a second user for treating and caring the individual during a second phase of discharge. In some instances, the second user is a healthcare professional.

### Suggestions

Described herein, in some embodiments, are systems, methods, software, and platforms for treating an individual by configuring a first processor and a first software to receive an identifier for the individual, to associate the identifier with a first phase of a plurality of phases of a medical care encounter experienced by the individual, and to provide to a first user a first suggestion for caring for the individual during the first phase of the plurality of phases of the medical care encounter experienced by the individual. In some instances, the systems, methods, software, and platforms for treating the individual comprise a second processor and a second software to receive the identifier for the individual, to associate the identifier with a second phase of the plurality of phases of the medical care encounter experienced by the individual, and to provide to a second user a second suggestion for caring for the individual during a second phase of the plurality of phases of the medical care encounter experienced by the individual. In some instances, the suggestions can be further modified for improvement such as compliance to the suggestions and outcomes of treating the patient based on the suggestions. In some cases, the improvement can be based on healthcare professionals tracking progress and outcome of the plurality of phases medical care encounter. In some cases, when improved outcome or progress are observed, healthcare professionals can report and record the improvement to the server. In some embodiments, when the improvement are observed, healthcare professionals can review the suggestions that yield the improvement.

In some cases, the suggestions and the outcome of the healthcare professionals treating the individuation based on the suggestions can be tracked, stored, and analyzed by a server or the software. In some cases, the server or the software can perform machine learning based analysis to identity possible improvements for the suggestions. In such instances, the healthcare professionals can confirm, modify or deny the suggestions.

Described herein, in some embodiments, are systems, methods, software, and platforms for providing suggestions for treating and caring an individual during a plurality of phases of medical care encounter experienced by the individual. In some cases, the suggestions can be at least partially determined by an identifier of the individual. In some instances, suggestions of one phase can be at least partially determined by data, results, or suggestions of other phases. In some embodiments, suggestions can be first suggestions or second suggestions. In some cases, suggestions can be both first suggestions and second suggestions in the same of different phases. In some embodiments, the first suggestions can at least partially determine the second suggestions. In some instances, the second suggestions can at least partially be determined by the first suggestions. In some embodiments, first suggestions are specific to a first user for caring and treating the individual during a first phase of the plurality of phases of medical care encounter. In some embodiments, second suggestions are specific to a second user for caring and treating the individual during a second phase of the plurality of phases of medical care encounter. In some cases, both first and second suggestions can be modified based on the data provided by the identifier. In some cases, the first suggestions are provided by a first processor and a first software. In some embodiments, the second suggestions are provided by a second processor and a second software.

In some cases, suggestions in exemplary format of diagnosis, protocols, instructions, illustration, warning, or checklist are provided to the first and second users during each phase of the plurality phases of medical encounter. In some instances, the suggestions can be provided to the first and second users through any computing device as described herein. In some cases, the suggestions can be provided via the first user portal, the second user porta, or any other user portals. In some embodiments, the suggestions can be provided to the users through visual, audio, or tactile means. In some cases, the suggestions can be displayed on a monitor of a computing device. In some embodiments, the suggestions can be displayed in any form of multi-media modes of communication such as text, graphics, video, audio, or any combination thereof.

In some cases, the suggestions can be protocols. In some embodiments, the protocols can be passive, i.e. a source of information which the healthcare professionals can choose to follow or consult. In some alternatives, the protocols can be active. Active protocols can provide specific guidelines for users or healthcare professionals to follow. In some cases, active protocols comprise checklist or oversight mechanism, where certain healthcare professionals can verify compliance of the protocols by other healthcare professionals. In some embodiments, the suggestion can be checklist. In some cases, the checklist must be checked and verified by users or other healthcare professionals who provide oversight.

In some instances, the suggestions can be prompts or alerts. In some embodiments, the prompts or alerts can be directed to users of the suggestions. In some cases, the prompts or alerts can be directed to healthcare professionals who are not users of the suggestions. In some cases, the prompts or alerts can be active at a predetermined time frame or time interval. In some embodiments, the prompts or alerts can be activated based on vital signs of the individual. In some embodiments, the prompts or alerts can be activated based on inputs of the individual or healthcare professionals. In some cases, the prompts or alerts can not be overridden.

In some embodiments, the suggestions provided by the software and processor can be directed to pediatric care. In some cases, the suggestions can be directed to users or individuals who are pediatric patients. In some instances, the suggestions directed for pediatric use can be configured to be more visual, comprising cartoons or illustrations for ease of interacting with the user.

### Pediatric Focus

In some embodiments, described herein are systems, methods, or platforms for treating an individual who is a pediatric patient. In some embodiments, the identifiers and suggestions for the individual who is a pediatric patient are at least partially determined by the individual's age, body weight, and stage of development. In some embodiments, the suggestions as described in this instant disclosure can be adopted or customized based on identifier of the individual as well as additional physical and psychological parameters directed to a pediatric patient. An exemplary list of physical parameters that contribute to the generation of suggestions for treating an individual who is a pediatric patient include children having: a higher body surface area than adults; a greater risk of losing body heat and fluids; thinner skin and absorbing toxin through skin more readily; cells dividing more rapidly; higher heart rate and respiratory rate; immature blood barrier, higher metabolic rate, smaller airways, smaller vasculature, and immature immune systems. An exemplary list of psychological parameters that contribute to the generation of suggestions for treating an individual who is a pediatric patient include children having: a more volatile development of social skills, emotional management, and cognition. In some instances, the suggestions comprise instructing healthcare professionals to examine exposures that are unique to children, e.g. transplacental and through breastfeeding. In some embodiments, the suggestions incorporate differences between how children and adults respond to drugs. Some of the differences of responding to drugs that can at least partially determine the suggestions include: drug absorption, drug distribution depending on percentage and distribution of bodily fluids, drug metabolism in liver, or drug elimination via liver and kidney. In some embodiments, the suggestions for pediatric patient comprises calculating a pediatric dosage as opposed to adjusting for a dosage based on an adult dosage for administering of therapeutics.

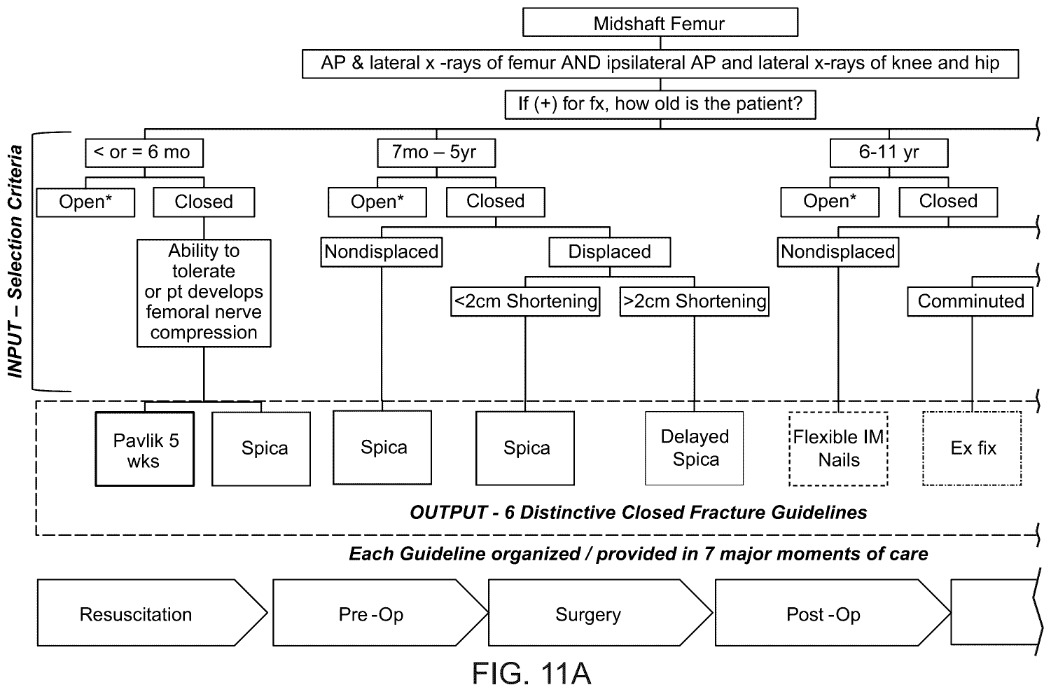

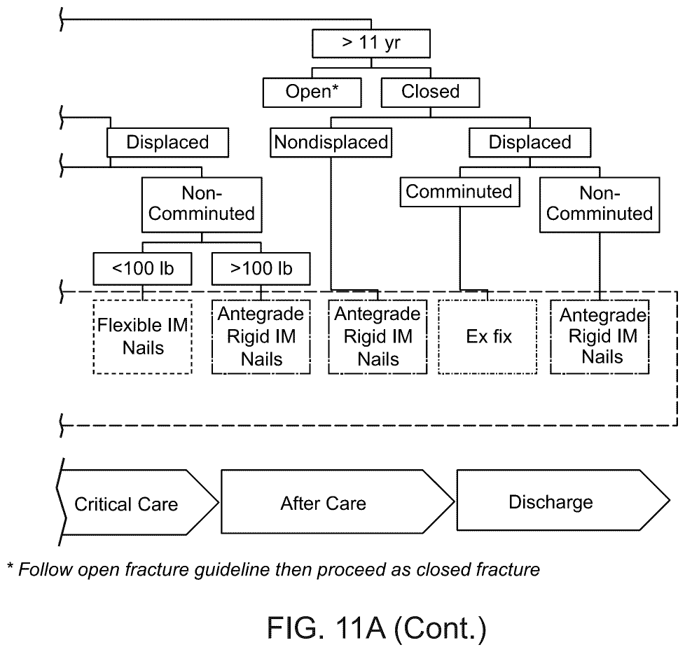

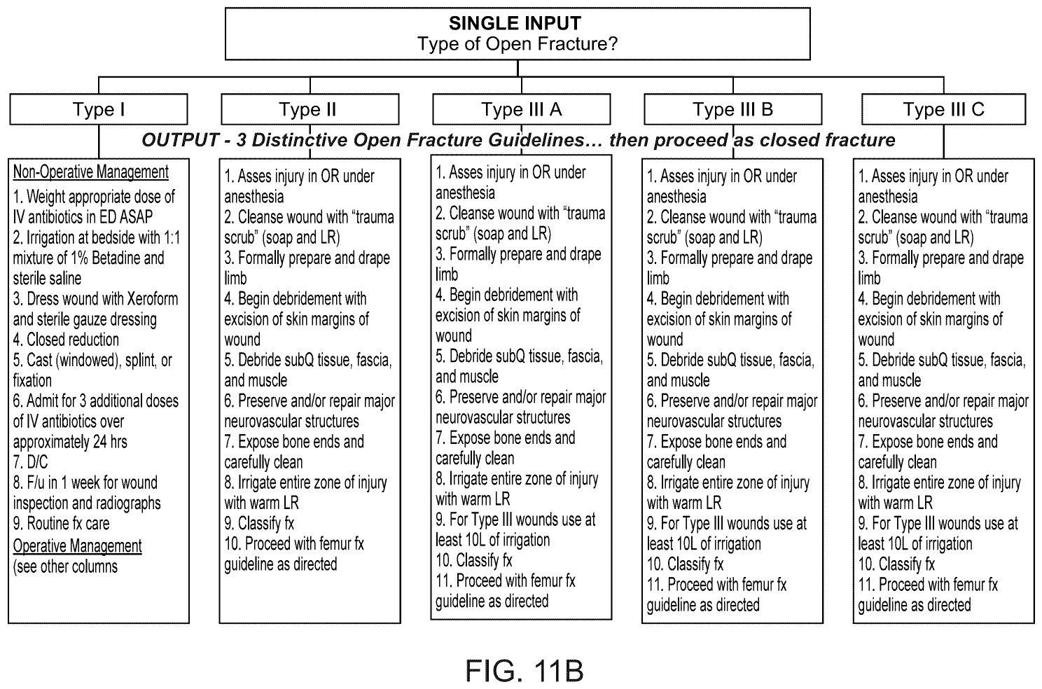

FIG. 11A provides an exemplary flowchart of how the systems, methods, software, and platforms of the instant disclosure provide suggestions based on the identifier of the individual who is a pediatric patient suffering from a closed femur fracture. Depending on the age of the individual, a different set of suggestions are provided for each phase of the plurality of phases of the medical care encounter. For example, if the individual is between 7-month to 5 year-old, spica casting may be delayed depending on the severity of the fracture. FIG. 11B illustrates how the systems. Methods, software, and platforms described herein provide suggestions in the form of sequential protocols to treat the individual is the femur fracture is an open femur fracture. Suggestions are at least partially determined and grouped based on the type of open femur fracture.

In some embodiments, the suggestions for treating and caring the individual who is a pediatric patient comprise consideration of normal behavior for the individual's age. In some embodiments, speech pattern and language skill can at least partially contribute to the suggestions. In some cases, the tracking and monitoring growth curve of the pediatric patient can be at least partially contributing to the suggestions. In some embodiments, the suggestions for treating and caring the individual who is a pediatric patient comprise consideration and inputs from healthcare professionals (e.g. healthcare professionals from social services, child protective services, psychiatry, psychology, children welfare, etc). In some embodiments, suggestions provided for treating the pediatric patient comprise additional inputs from protocols and inputs of specialties medicine including: adolescent medicine, child abuse pediatrics, clinical informatics, developmental-behavioral pediatrics, genetics, headache medicine, hospice and palliative care, medical toxicology, neonatology, pain medicine, pediatric allergy and immunology, pediatric cardiology, pediatric cardiac critical care, pediatric critical care, pediatric emergency medicine, pediatric endocrinology, pediatric gastroenterology, transplant hepatology, pediatric hematology, pediatric infectious disease, pediatric nephrology, pediatric oncology, pediatric neuro-oncology, pediatric pulmonology, sleep medicine, pediatric rheumatology, social pediatrics, sports medicine, child neurology, brain injury medicine, clinical neurophysiology, endovascular neuroradiology, epilepsy, neurocritical care, neuroimmunology, neuromuscular medicine, vascular neurology, child psychiatry, neurodevelopmental disabilities, pediatric anesthesiology, pediatric dentistry, pediatric dermatology, pediatric gynecology, pediatric neurosurgery, pediatric ophthalmology, pediatric orthopedic surgery, pediatric otolaryngology, pediatric radiology, pediatric rehabilitation medicine, pediatric surgery, or pediatric urology.

In some instances, the identifiers and suggestions as provided by the systems, methods, software, and platforms can be displayed as part of a web application or mobile application on any of the computing devices or user portals as described herein. In some embodiments, the identifiers suggestions as displayed can be further modified and customized to for ease of use by an individual or user who is a pediatric patient. In some embodiments, the identifiers or suggestions comprise pictorial depictions, cartoons, or illustrations that aid in communications among users.

### Identifier

In certain aspects, the systems, methods, software, and platforms disclosed herein receive, generate, and analyze the identifier comprising data, information, or profile of the individuals being treated by the healthcare professionals, inputs provided by both the individual and the healthcare professional, and electronic medical records. In some embodiments, the identifier at least partially determines suggestions of the phases of the plurality of phases of the medical care encounter. In some embodiments, the identifier can be modified, analyzed, monitored, and tracked.

In some embodiments, the identifier comprises personal and medial information of the individual such as age, sex or gender, race or ethnicity, weight, height, body mass index (BMI), heart rate (e.g. ECG and/or peripheral pulse rate), blood pressure, body temperature, respiration rate, past checkups, treatments or therapies, drugs administered, observations, vaccinations, current and/or past symptoms (e.g. fever, vomiting, cough, etc.), known health conditions (e.g. allergies), known diseases or disorders, health history (e.g. past diagnoses), lab test results (e.g. blood test), lab imaging results (e.g. X-rays, MRIs, etc.), genetic information (e.g. known genetic abnormalities associated with disease), family medical history, or any combination thereof. In some embodiments, physiological parameters of the individual is being monitored, recorded, and analyzed based on signals generated by one or more sensor and processors. In some cases, the identifier comprises records of treatments already administered to the individuals. In some embodiments, the identifier comprises inputs, suggestions, or protocols from one healthcare professional to another healthcare professional. In some embodiments, the identifier can be tracked and updated over time. In some embodiments, the identifier can be analyzed for improvements in generating subsequent suggestions.

In some embodiments, the identifier comprises inputs provided by the individual. In some cases, the identifier comprises inputs provided by the healthcare professionals or both the healthcare professional and the individual. In some instances, the inputs comprise observations made by the healthcare professionals. In some cases, the inputs comprise self-assessment such as pain or discomfort level of the individual. In some cases, the inputs comprise observation and assessment made by both the individual and healthcare professionals. In some embodiments, the inputs comprises communications between the individual and the healthcare professionals or between healthcare professionals. In some cases, the inputs comprise direction, instruction, or warning provided by the healthcare professionals. In some instances, inputs comprise usage and oversight as recorded from the users using the systems, methods, software, and platforms. Exemplary oversight includes how diligent and how frequent users follow the suggestions and how often users override the suggestions. In some embodiments, the inputs can be tracked and updated over time. In some embodiments, the inputs can be analyzed for improvements in generating subsequent suggestions.

In some instances, the identifier comprises electronic medical record. Electronic medical record can be any result, record, documentation, and any other relevant information pertaining to the medical care encounter of the individual. Non-limiting example of electronic medical record includes digitals version of medical charts. In some instances, the electronic medical record comprises personal, medical, and treatment history of the individuals being treated. In some embodiments, the electronic medical record can be tracked and updated over time. In some embodiments, the electronic medical record can be analyzed for improvements in generating subsequent suggestions.

### Computing System

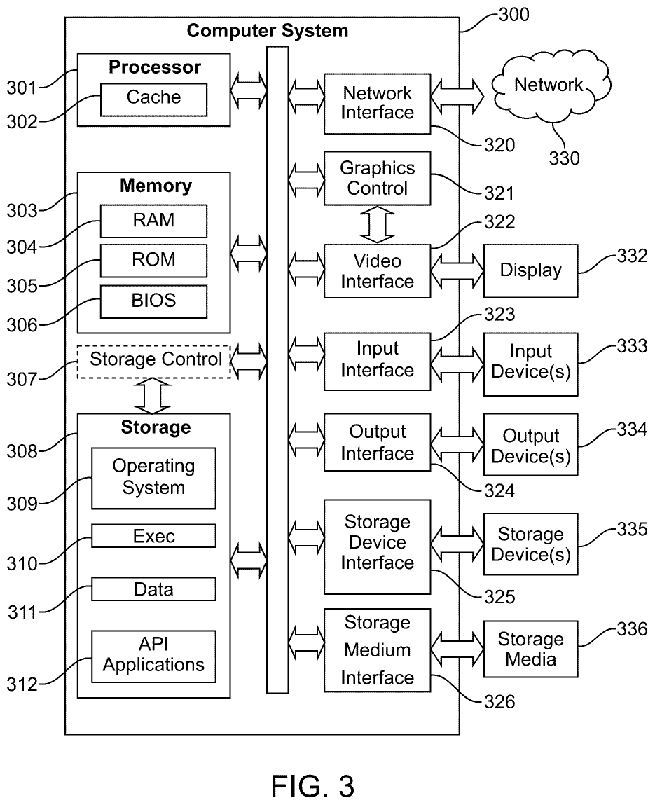

In some embodiments, the systems, methods, software, and platforms as described herein comprise use of a computer system. Referring to FIG. 3, a block diagram is shown depicting an exemplary machine that includes a computer system 300 (e.g., a processing or computing system) within which a set of instructions can execute for causing a device to perform or execute any one or more of the aspects and/or methodologies for static code scheduling of the present disclosure. The components in FIG. 3 are examples only and do not limit the scope of use or functionality of any hardware, software, embedded logic component, or a combination of two or more such components implementing particular embodiments.

Computer system 300 can include one or more processors 301, a memory 303, and a storage 308 that communicate with each other, and with other components, via a bus 340. The bus 340 can also link a display 332, one or more input devices 333 (which may, for example, include a keypad, a keyboard, a mouse, a stylus, etc.), one or more output devices 334, one or more storage devices 335, and various tangible storage media 336. All of these elements can interface directly or via one or more interfaces or adaptors to the bus 340. For instance, the various tangible storage media 336 can interface with the bus 340 via storage medium interface 326. Computer system 300 can have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

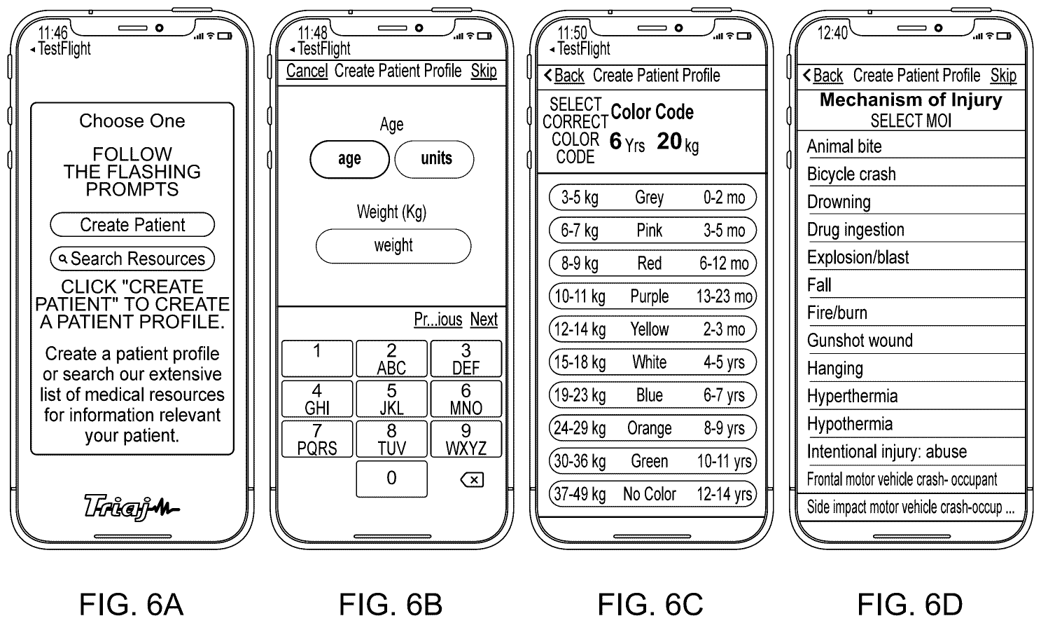

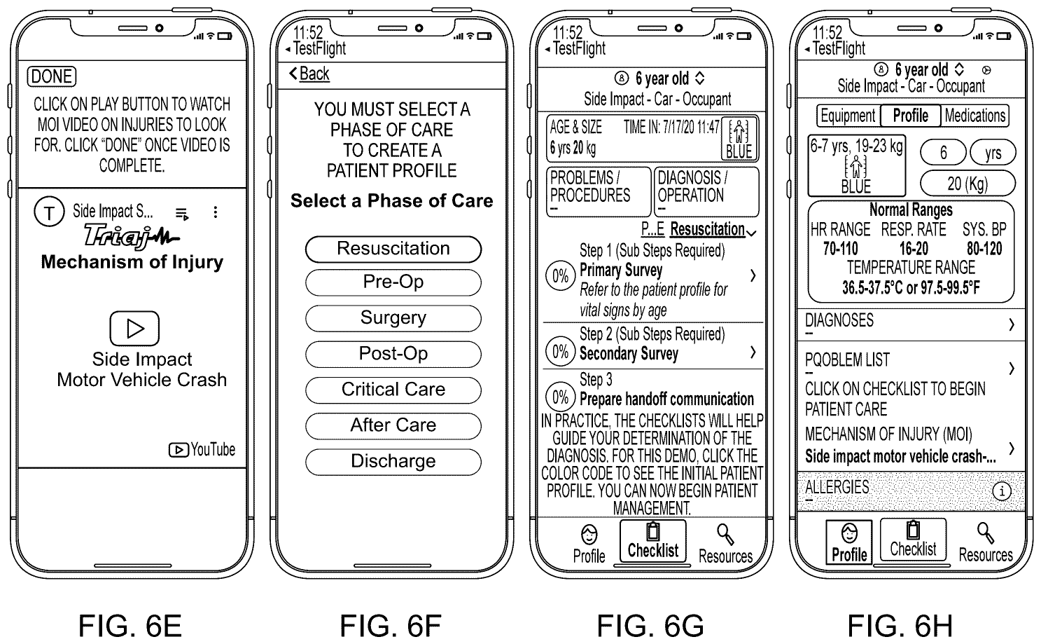

Computer system 300 includes one or more processor(s) 301 (e.g., central processing units (CPUs) or general purpose graphics processing units (GPGPUs)) that carry out functions. Processor(s) 301 optionally contains a cache memory unit 302 for temporary local storage of instructions, data, or computer addresses. Processor(s) 301 are configured to assist in execution of computer readable instructions. Computer system 300 can provide functionality for the depictions in FIGS. 6-8 as a result of the processor(s) 301 executing non-transitory, processor-executable instructions embodied in one or more tangible computer-readable storage media, such as memory 303, storage 308, storage devices 335, and/or storage medium 336. The computer-readable media can store software that implements particular embodiments, and processor(s) 301 can execute the software. Memory 303 can read the software from one or more other computer-readable media (such as mass storage device(s) 335, 336) or from one or more other sources through a suitable interface, such as network interface 320. The software can cause processor(s) 301 to carry out one or more processes or one or more steps of one or more processes described or illustrated herein. Carrying out such processes or steps can include defining data structures stored in memory 303 and modifying the data structures as directed by the software.

The memory 303 can include various components (e.g., machine readable media) including, but not limited to, a random access memory component (e.g., RAM 304) (e.g., static RAM (SRAM), dynamic RAM (DRAM), ferroelectric random access memory (FRAM), phase-change random access memory (PRAM), etc.), a read-only memory component (e.g., ROM 305), and any combinations thereof. ROM 305 can act to communicate data and instructions unidirectionally to processor(s) 301, and RAM 304 can act to communicate data and instructions bidirectionally with processor(s) 301. ROM 305 and RAM 304 can include any suitable tangible computer-readable media described below. In one example, a basic input/output system 306 (BIOS), including basic routines that help to transfer information between elements within computer system 300, such as during start-up, can be stored in the memory 303.

Fixed storage 308 is connected bidirectionally to processor(s) 301, optionally through storage control unit 307. Fixed storage 308 provides additional data storage capacity and can also include any suitable tangible computer-readable media described herein. Storage 308 can be used to store operating system 309, executable(s) 310, data 311, applications 312 (application programs), and the like. Storage 308 can also include an optical disk drive, a solid-state memory device (e.g., flash-based systems), or a combination of any of the above. Information in storage 308 may, in appropriate cases, be incorporated as virtual memory in memory 303.

In one example, storage device(s) 335 can be removably interfaced with computer system 300 (e.g., via an external port connector (not shown)) via a storage device interface 325. Particularly, storage device(s) 335 and an associated machine-readable medium can provide non-volatile and/or volatile storage of machine-readable instructions, data structures, program modules, and/or other data for the computer system 300. In one example, software can reside, completely or partially, within a machine-readable medium on storage device(s) 335. In another example, software can reside, completely or partially, within processor(s) 301.

Bus 340 connects a wide variety of subsystems. Herein, reference to a bus can encompass one or more digital signal lines serving a common function, where appropriate. Bus 340 can be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 300 can also include an input device 333. In one example, a user of computer system 300 can enter commands and/or other information into computer system 300 via input device(s) 333. Examples of an input device(s) 333 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 333 can be interfaced to bus 340 via any of a variety of input interfaces 323 (e.g., input interface 323) including, but not limited to, serial, parallel, game port, USB, FIREWIRE, THUNDERBOLT, or any combination of the above.

In particular embodiments, when computer system 300 is connected to network 330, computer system 300 can communicate with other devices, specifically mobile devices and enterprise systems, distributed computing systems, cloud storage systems, cloud computing systems, and the like, connected to network 330. Communications to and from computer system 300 can be sent through network interface 320. For example, network interface 320 can receive incoming communications (such as requests or responses from other devices) in the form of one or more packets (such as Internet Protocol (IP) packets) from network 330, and computer system 300 can store the incoming communications in memory 303 for processing. Computer system 300 can similarly store outgoing communications (such as requests or responses to other devices) in the form of one or more packets in memory 303 and communicated to network 330 from network interface 320. Processor(s) 301 can access these communication packets stored in memory 303 for processing.

Examples of the network interface 320 include, but are not limited to, a network interface card, a modem, and any combination thereof. Examples of a network 330 or network segment 330 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 330, can employ a wired and/or a wireless mode of communication. In general, any network topology can be used.

Information and data can be displayed through a display 332. Examples of a display 332 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 332 can interface to the processor(s) 301, memory 303, and fixed storage 308, as well as other devices, such as input device(s) 333, via the bus 340. The display 332 is linked to the bus 340 via a video interface 322, and transport of data between the display 332 and the bus 340 can be controlled via the graphics control 321. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 332, computer system 300 can include one or more other peripheral output devices 334 including, but not limited to, an audio speaker, a printer, a storage device, and any combinations thereof. Such peripheral output devices can be connected to the bus 340 via an output interface 324. Examples of an output interface 324 include, but are not limited to, a serial port, a parallel connection, a USB port, a FIREWIRE port, a THUNDERBOLT port, and any combinations thereof.

In addition or as an alternative, computer system 300 can provide functionality as a result of logic hardwired or otherwise embodied in a circuit, which can operate in place of or together with software to execute one or more processes or one or more steps of one or more processes described or illustrated herein. Reference to software in this disclosure can encompass logic, and reference to logic can encompass software. Moreover, reference to a computer-readable medium can encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein can be embodied directly in hardware, in a software module executed by one or more processor(s), or in a combination of the two. A software module can reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium can be integral to the processor. The processor and the storage medium can reside in an ASIC. The ASIC can reside in a user terminal. In the alternative, the processor and the storage medium can reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

### Non-Transitory Computer Readable Storage Medium

In some embodiments, the platforms, systems, media, software, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

### Computer Program

In some embodiments, the platforms, systems, media, software, and methods disclosed herein include at least one computer program, or use of the same. A computer program includes a sequence of instructions, executable by one or more processor(s) of the computing device's CPU, written to perform a specified task. Computer readable instructions can be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program can be written in various versions of various languages.

The functionality of the computer readable instructions can be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application can be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

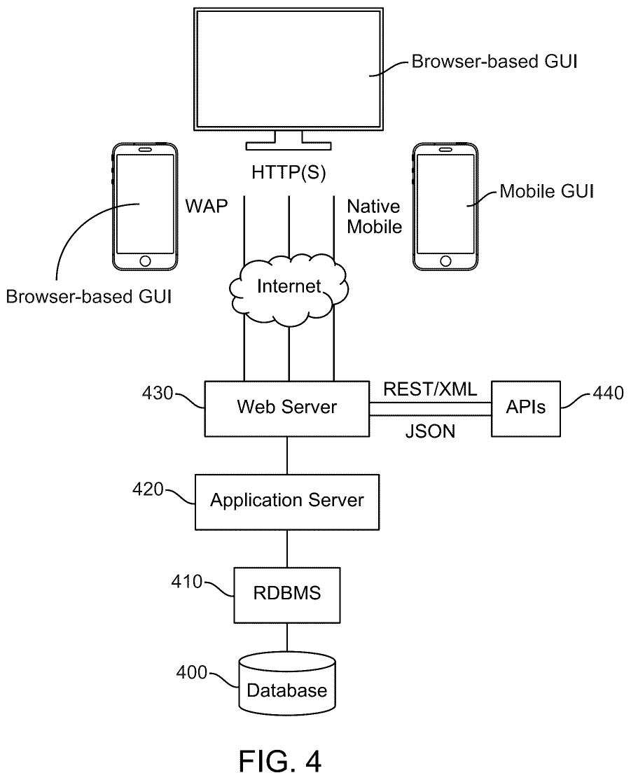

Referring to FIG. 4, in a particular embodiment, an application provision system comprises one or more databases 400 accessed by a relational database management system (RDBMS) 410. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 420 (such as Java servers, .NET servers, PHP servers, and the like) and one or more web servers 430 (such as Apache, IIS, GWS and the like). The web server(s) optionally expose one or more web services via app application programming interfaces (APIs) 440. Via a network, such as the Internet, the system provides browser-based and/or mobile native user interfaces.

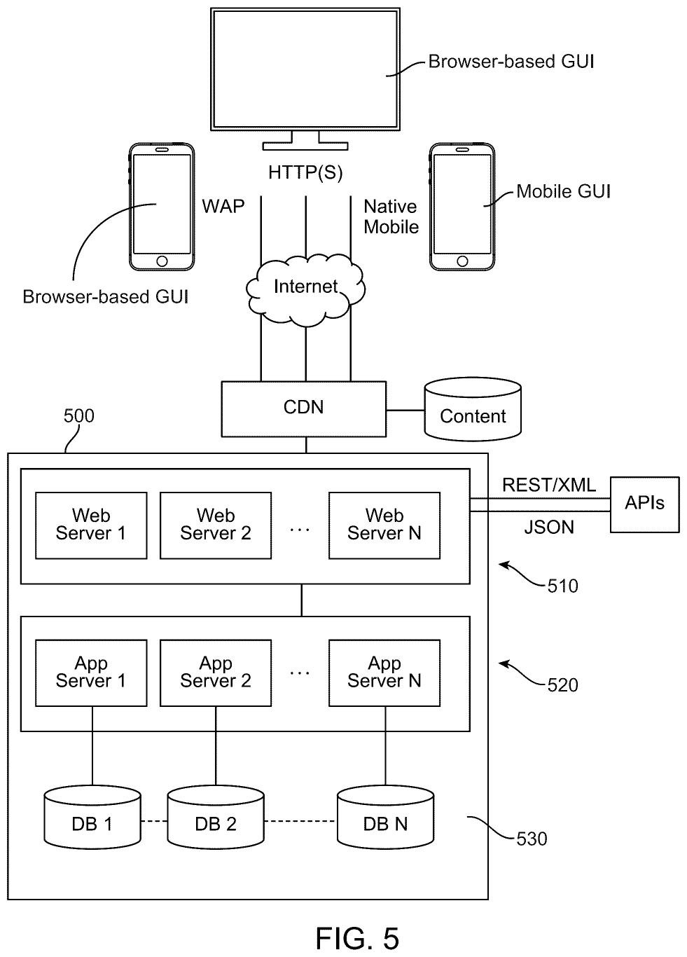

Referring to FIG. 5, in a particular embodiment, an application provision system alternatively has a distributed, cloud-based architecture 500 and comprises elastically load balanced, auto-scaling web server resources 510 and application server resources 520 as well synchronously replicated databases 530.

### Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite,.NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB .NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

### Databases

In some embodiments, the platforms, systems, media, software, and methods disclosed herein include one or more databases, or use of the same. In view of the disclosure provided herein, those of skill in the art will recognize that many databases are suitable for storage and retrieval of information related to the systems, methods, software, and platforms as described herein. In various embodiments, suitable databases include, by way of non-limiting examples, relational databases, non-relational databases, object oriented databases, object databases, entity-relationship model databases, associative databases, and XML databases. Further non-limiting examples include SQL, PostgreSQL, MySQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

### Methods Utilizing a Computer

The methods and software described herein can utilize one or more computers. The computer can be used for managing customer and sample information such as sample or customer tracking, database management, analyzing molecular profiling data, analyzing cytological data, storing data, billing, marketing, reporting results, storing results, or a combination thereof. The computer can include a monitor or other graphical interface for displaying data, results, billing information, marketing information (e.g. demographics), customer information, or sample information. The computer can also include means for data or information input. The computer can include a processing unit and fixed or removable media or a combination thereof. The computer can be accessed by a user in physical proximity to the computer, for example via a keyboard and/or mouse, or by a user that does not necessarily have access to the physical computer through a communication medium such as a modem, an internet connection, a telephone connection, or a wired or wireless communication signal carrier wave. In some cases, the computer can be connected to a server or other communication device for relaying information from a user to the computer or from the computer to a user. In some cases, the user can store data or information obtained from the computer through a communication medium on media, such as removable media. It is envisioned that data relating to the methods can be transmitted over such networks or connections for reception and/or review by a party. The receiving party can be but is not limited to an individual, a health care provider or a health care manager. In one instance, a computer-readable medium includes a medium suitable for transmission of a result of an analysis of a biological sample. The medium can include a result of a subject, wherein such a result is derived using the methods described herein.

The entity obtaining the sample information can enter it into a database for the purpose of one or more of the following: inventory tracking, assay result tracking, order tracking, customer management, customer service, billing, and sales. Sample information can include, but is not limited to: customer name, unique customer identification, customer associated medical professional, indicated assay or assays, assay results, adequacy status, indicated adequacy tests, medical history of the individual, preliminary diagnosis, suspected diagnosis, sample history, insurance provider, medical provider, third party testing center or any information suitable for storage in a database. Sample history can include but is not limited to: age of the sample, type of sample, method of acquisition, method of storage, or method of transport.

The database can be accessible by a customer, medical professional, insurance provider, or other third party. Database access can take the form of digital processing communication such as a computer or telephone. The database can be accessed through an intermediary such as a customer service representative, business representative, consultant, independent testing center, or medical professional. The availability or degree of database access or sample information, such as assay results, can change upon payment of a fee for products and services rendered or to be rendered. The degree of database access or sample information can be restricted to comply with generally accepted or legal requirements for patient or customer confidentiality.

### Machine Learning

The systems, methods, software, and platforms as described herein can comprise computer-implemented methods of supervised or unsupervised learning methods, including SVM, random forests, clustering algorithm (or software module), gradient boosting, logistic regression, and/or decision trees. The machine learning methods as described herein can improve generation of suggestions based on recording and analyzing any of the identifiers, lab results, patient outcomes, or any other relevant medical information as described herein. In some cases, the machine learning methods can intentionally group or separate treatment options. In some embodiments, some treatment options can be intentionally clustered or removed from any one phase of the plurality of phases of the medical care encounter.

Supervised learning algorithms can be algorithms that rely on the use of a set of labeled, paired training data examples to infer the relationship between an input data and output data. Unsupervised learning algorithms can be algorithms used to draw inferences from training data sets to output data. Unsupervised learning algorithms can comprise cluster analysis, which can be used for exploratory data analysis to find hidden patterns or groupings in process data. One example of an unsupervised learning method can comprise principal component analysis. Principal component analysis can comprise reducing the dimensionality of one or more variables. The dimensionality of a given variables can be at least 1, 5, 10, 50, 100, 200, 300, 400, 500, 600, 700, 800, 900, 1000, 1100, 1200 1300, 1400, 1500, 1600, 1700, 1800, or greater. The dimensionality of a given variables can be at most 1800, 1600, 1500, 1400, 1300, 1200, 1100, 1000, 900, 800, 700, 600, 500, 400, 300, 200, 100, 50, 10 or less.

The computer-implemented methods can comprise statistical techniques. In some embodiments, statistical techniques can comprise linear regression, classification, resampling methods, subset selection, shrinkage, dimension reduction, nonlinear models, tree-based methods, support vector machines, unsupervised learning, or any combination thereof.

A linear regression can be a method to predict a target variable by fitting the best linear relationship between a dependent and independent variable. The best fit can mean that the sum of all distances between a shape and actual observations at each point is the least. Linear regression can comprise simple linear regression and multiple linear regression. A simple linear regression can use a single independent variable to predict a dependent variable. A multiple linear regression can use more than one independent variable to predict a dependent variable by fitting a best linear relationship.

A classification can be a data mining technique that assigns categories to a collection of data in order to achieve accurate predictions and analysis. Classification techniques can comprise logistic regression and discriminant analysis. Logistic regression can be used when a dependent variable is dichotomous (binary). Logistic regression can be used to discover and describe a relationship between one dependent binary variable and one or more nominal, ordinal, interval or ratio-level independent variables. A resampling can be a method comprising drawing repeated samples from original data samples. A resampling can not involve a utilization of a generic distribution tables in order to compute approximate probability values. A resampling can generate a unique sampling distribution on a basis of an actual data. In some embodiments, a resampling can use experimental methods, rather than analytical methods, to generate a unique sampling distribution. Resampling techniques can comprise bootstrapping and cross-validation. Bootstrapping can be performed by sampling with replacement from original data and take “not chosen” data points as test cases. Cross validation can be performed by split training data into a plurality of parts.

A subset selection can identify a subset of predictors related to a response. A subset selection can comprise best-subset selection, forward stepwise selection, backward stepwise selection, hybrid method, or any combination thereof. In some instances, shrinkage fits a model involving all predictors, but estimated coefficients are shrunken towards zero relative to the least squares estimates. This shrinkage can reduce variance. A shrinkage can comprise ridge regression and a lasso. A dimension reduction can reduce a problem of estimating n+1 coefficients to a simpler problem of m+1 coefficients, where m<n. It can be attained by computing n different linear combinations, or projections, of variables. Then these n projections are used as predictors to fit a linear regression model by least squares. Dimension reduction can comprise principal component regression and partial least squares. A principal component regression can be used to derive a low dimensional set of features from a large set of variables. A principal component used in a principal component regression can capture the most variance in data using linear combinations of data in subsequently orthogonal directions. The partial least squares can be a supervised alternative to principal component regression because partial least squares can make use of a response variable in order to identify new features.

A nonlinear regression can be a form of regression analysis in which observational data are modeled by a function which is a nonlinear combination of model parameters and depends on one or more independent variables. A nonlinear regression can comprise a step function, piecewise function, spline, generalized additive model, or any combination thereof.

Tree-based methods can be used for both regression and classification problems. Regression and classification problems can involve stratifying or segmenting the predictor space into a number of simple regions. Tree-based methods can comprise bagging, boosting, random forest, or any combination thereof. Bagging can decrease a variance of prediction by generating additional data for training from the original dataset using combinations with repetitions to produce multistep of the same carnality/size as original data. Boosting can calculate an output using several different models and then average a result using a weighted average approach. A random forest algorithm can draw random bootstrap samples of a training set. Support vector machines can be classification techniques. Support vector machines can comprise finding a hyperplane that best separates two classes of points with the maximum margin. Support vector machines can constrain an optimization problem such that a margin is maximized subject to a constraint that it perfectly classifies data.

Unsupervised methods can be methods to draw inferences from datasets comprising input data without labeled responses. Unsupervised methods can comprise clustering, principal component analysis, k-Mean clustering, hierarchical clustering, or any combination thereof.

### Graphic User Interface (GUI)

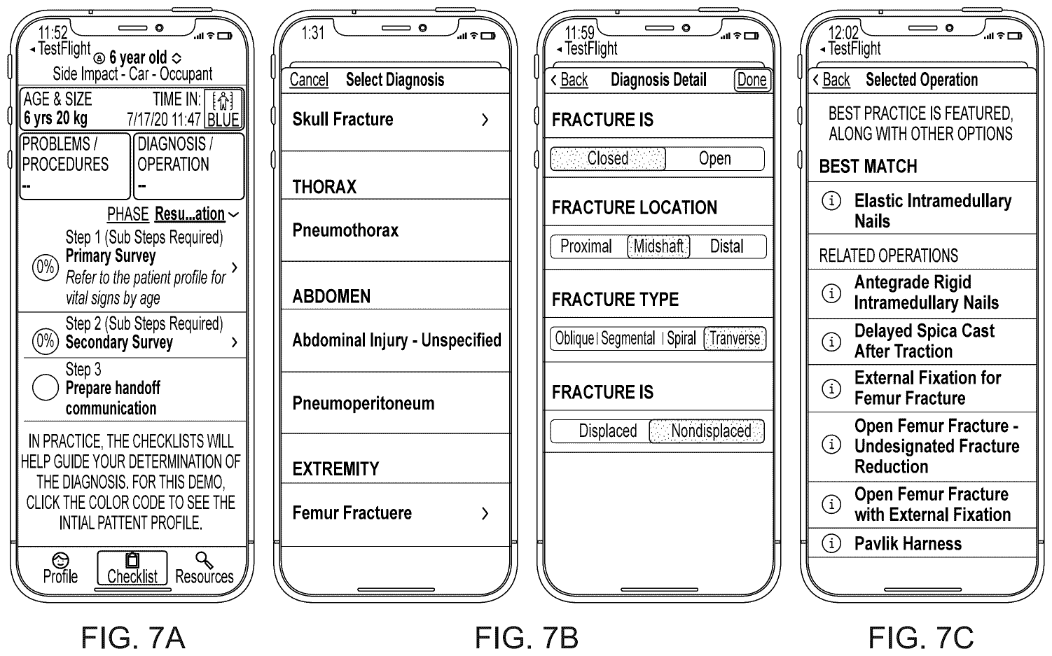

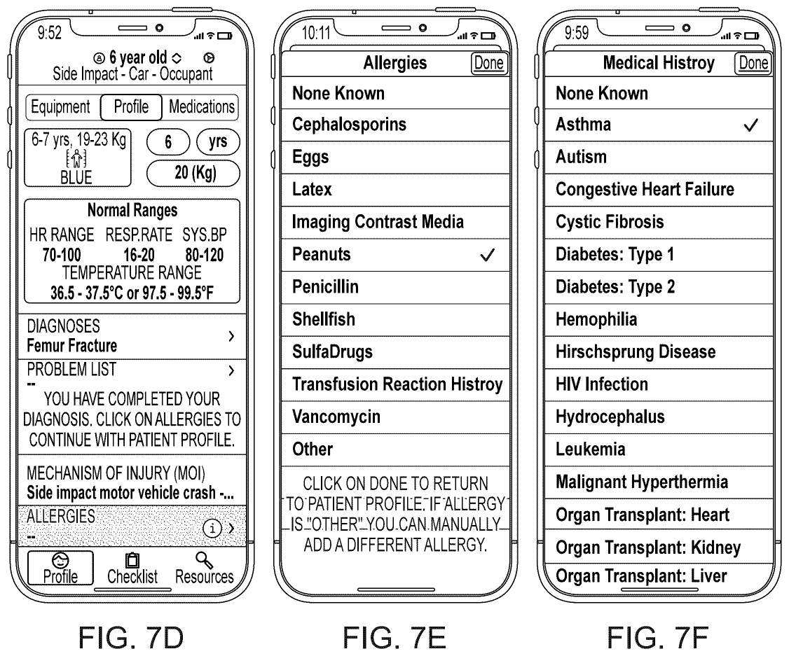

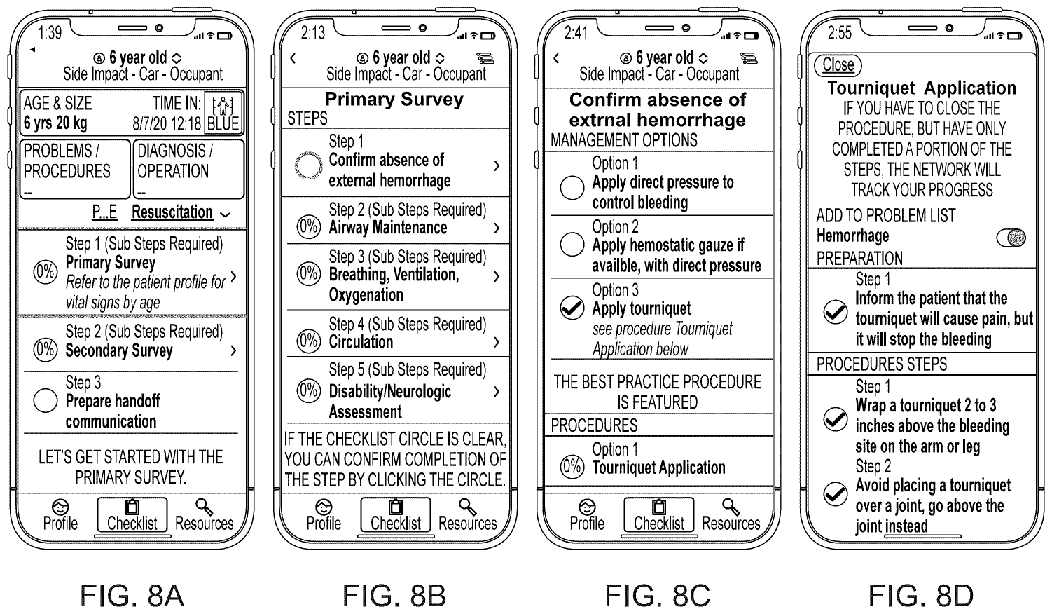

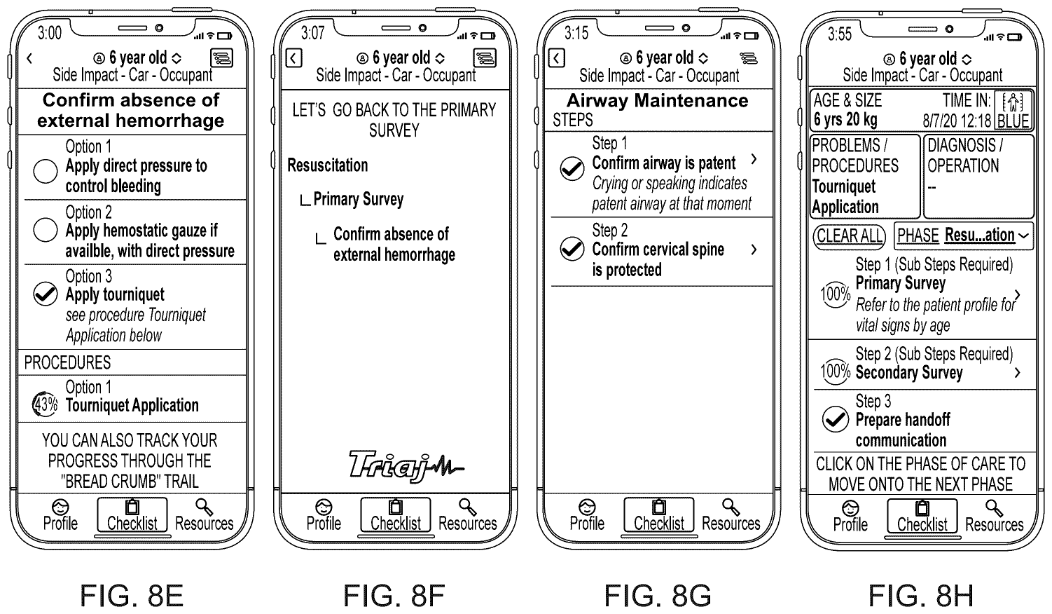

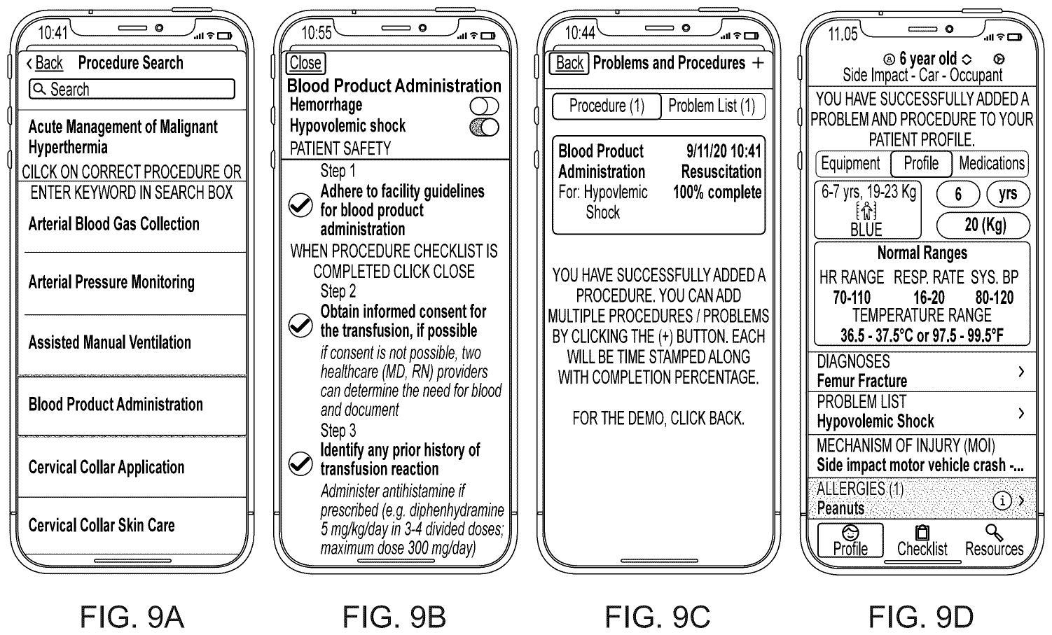

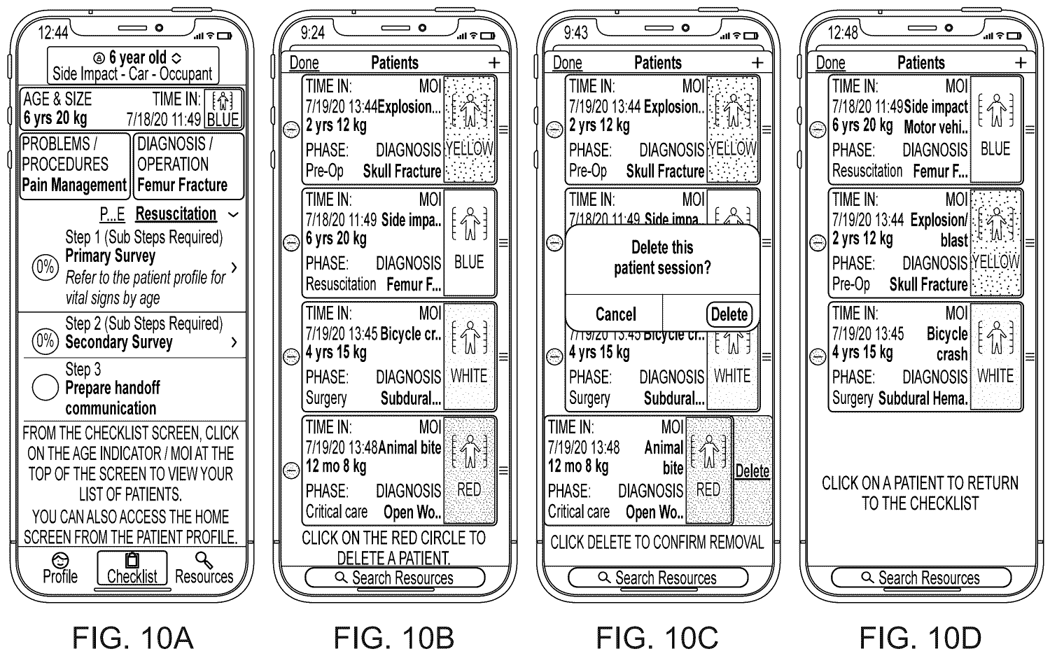

In some embodiments, the systems, methods, software, and platforms described herein can comprise means of communications and interacting with the users through a graphic user interface. In some embodiments, the graphic user interface depicts the software as described herein. In some instances, the graphic user interface can be displayed on any of the computing device and user portals. In some embodiments, the graphic user interface can be customized for a user who is a pediatric patient. In some embodiments, the graphic user interface can comprise multiple modules comprising: start up and onboarding, session management, diagnosis, patient profile and mechanism of injury, general checklist structure, phases of care, alert messages, and search function. In some embodiments, the graphic user interface can present a series of questions for a user to answer. In some cases, the user can be an individual who is not a healthcare professional or a healthcare professional. In some embodiments, the graphic user interface can depict graphic or cartoon illustration of a human body. In some embodiments, the graphic user interface can alert the users with additional pop-up messages or any other form of communications. FIG. 6-10 illustrate exemplary depictions of the graphic user interface for receiving and displaying information and suggestions to the users to treat and care for a subject. FIG. 6A guides an user to create a patient profile or search for other resources. FIG. 6B and FIG. 6C show that the graphic user interface allow the user to enter age and weight. FIG. 6D shows selection of the mechanism of injury through the graphic user interface. FIG. 6E illustrates an exemplary video directed to the mechanism of injury selected: side impact from motor vehicle crash. FIG. 6F-G illustrate selection of phase of care for resuscitation. FIG. 7 illustrates completion of patient profile. FIG. 7A illustrates a checklist for the patient who is being treated. FIG. 7B illustrates screen shots for diagnosis selection of a femur fracture for the checklist. FIG. 7C illustrates operation or procedure selection based on the femur fracture selection from FIG. 7B. FIG. 7D illustrates selection of allergies (FIG. 7E) and medical history (FIG. 7F) prior to the operation or procedure. FIG. 8 illustrates a guide for checklist navigation. . . . FIG. 8A-C illustrate primary survey selection, where a tourniquet is selected based on the input (FIG. 8D). FIG. 8E illustrates procedures or operations directed to application of tourniquet. FIG. 8F illustrates instruction to confirm absence of external hemorrhage. FIG. 8G illustrates instruction to confirm that the patient's airway is not obstructed. FIG. 8H illustrates completion of the checklist of the web application. FIG. 9 illustrates exemplary screen captures of graphic user interface of a web application displayed on a computing device, where the users interact and utilize the systems, methods, software, and platforms as described herein for searching procedures to treat and care for an individual. FIG. 9A-C illustrate procedure search selection, where blood product administration is chosen and displayed. FIG. 9A illustrates a list of procedures to be selected by the user. FIG. 9B illustrates the product for blood product administration. FIG. 9C illustrates confirmation for adding the blood product administration. FIG. 9D illustrates completion of the procedure search. FIG. 10 illustrates exemplary screen captures of graphic user interface of a web application displayed on a computing device, where the users interact and utilize the systems, methods, software, and platforms as described herein to prioritize a patient session to treat and care for an individual. FIG. 10A illustrates a completed patient profile. FIG. 10B illustrates multiple patient session profiles. FIG. 10C illustrates a patient session being confirmed selected for deletion, and FIG. 10D illustrates a screen capture of confirmation of a patient session being deleted.

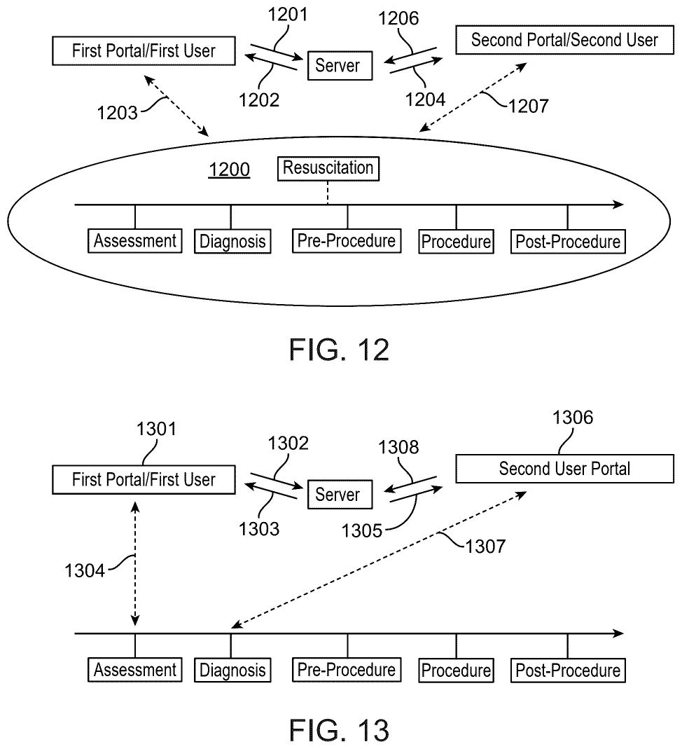

FIG. 12 illustrates an exemplary work flow of a first user and a second user utilizing suggestions provided by the systems, methods, software, and platforms as described herein to care and treat an individual through a plurality phases of medical care encounter 1200 experienced by the individual. A first user enters medical and personal information of the individual into the first portal. The first portal transmits the entered information of the individual 1201 to a server. The server records and analyzes the information and provides a suggestion back to the first user either directly 1202 or through the first portal. The first user utilizes the suggestion to care and treat the individual for a first phase of the plurality of phases 1203 medical care encounter. In some cases, the first user enters additional information or observation gathered from the plurality of phases of caring and treating the individual through the first portal to the server 1203 and 1201. The server then generates a second suggestion and transmits the second suggestion to a second user via second portal 1204. The second user can make changes to the suggestion by communicating back 1206 to the server or utilize the second suggestion to treat the individual for a second phase 1207 of the plurality of phases of medical care encounter. In some cases, the second user can provide server with additional medical information based on observation and results of the treatment of the second phase 1207 and 1206.

In some embodiments, the systems, methods, software, and platforms described herein allow an user of to search for resources. The resources can include text (e.g. about 250 words), images, or videos. In some embodiments, the GUI allows access through bottom tab bar in patient session or allow going directly to resources after opening the web/mobile application. In some embodiments, the resources can be identified where appropriate from the checklists. In some embodiments, the GUI allows scrolling through all resources. In some embodiments, the GUI allows typing in key word (letters) to find content more quickly. In some embodiments, the GUI comprises expansion of diagnosis and operation functions. In some embodiments, the GUI allows the screens of the computing devices for selection of diagnosis changed to a list by body region rather than the figure of the child. In some embodiments, the GUI allows selection of a diagnosis then offers the opportunity to select an operation. In some embodiments, the GUI displays a best match and lists related operations. In some embodiments, the web/mobile application allows the information button to provide access to the steps of the operation. In some embodiments, the GUI allows multiple diagnoses and operations can now be tracked in one patient. In some embodiments, the GUI allows searching for a specific procedure from within the patient session or checklist screen. In some embodiments, the web/mobile application allows the procedure selected to be specifically tied to a patient. In some embodiments, the GUI displays the problem associated with (or reason procedure is being performed) selected on the Procedure checklist. In some embodiments, the procedure checklist can be clicked to document progress in performing the procedure. In some embodiments, the procedure status (along with problem and date/time stamp) is documented on the Procedure/Problem screen. In some embodiments, the procedure can be documented on the patient profile. In some embodiments, the web/mobile application allows access a procedure in progress or to go back and restart a procedure. In some embodiments, the web/mobile application allows procedure steps that utilizes sequential performance of the procedure. In some embodiments, the web/mobile application connects the navigation of the web/mobile application on a mobile computing device to a website. In some embodiments, the web/mobile application connects the navigation demonstration of the web/mobile application to a website. In some embodiments, the web/mobile application allows natural Language Processing to create a “hands-free” verbal interaction between the web/mobile application and a healthcare provider. In some embodiments, the web/mobile application comprises or is connected to active learning recurring neural network, where the active learning recurring neural network actively seeks out anomalies in the care pathways of a specific diagnosis. In some embodiments, the web/mobile application comprises or is connected to the Electronic Health Record (EHR), enabling measuring outcomes within the systems, software, or platforms described herein. to evaluate and evolve the best practice in real time. In some embodiments, the web/mobile application comprises suggestions for the next step in patient assessment, care processes, and treatment throughout all phases of care. This is especially important for young physician residents or newly employed nurses who typically do not function across all 7 phases of care. This allows them to learn in real time while employing the best practice methods. In some embodiments, the web/mobile application provides sharing of provider-to-provider information regarding a specific patient. Multiple medical care facilities can link to systems, software, and platforms in a modular manner. In some embodiments, the systems, methods, software, and platforms described herein enable sharing of best practices across the hospital network, accessed by all web/mobile application users within a medical care facility network (e.g., the medical care facility network may have one preferred practice guideline that needs to be uniformly followed by all hospitals in the network). This will set the way healthcare professionals care for patients and will reduce discrepancies with insurance companies,

### EXAMPLES

### Example 1. EMT Providing Immediate Assistance to a 9-Year-Old Complaining Abdominal Pain

A parent dials for emergency medical assistance when her 9-year-old is incapacitated by a sudden bout of abdominal pain. Healthcare professionals comprising EMT personnel are dispatched to the residence of the 9-year-old. EMT performs initial check-up of the 9-year-old by communicating with both the parent and the 9-year-old. EMT enters any relevant medical and personal information into a first user portal in a form of a mobile electronic tablet 1301 as shown in FIG. 13. The information entered forms the basis of an identifier associated with the 9-year-old. The first user portal transmits the identifier to a server 1302, which further analyzes the information of the identifier. The server transmits a first suggestion back 1303 to the first user portal to instruct the EMT on how to properly conduct an assessment phase of a plurality of phases of medical care encounter that the 9-year-old would experience. The EMT follows the first suggestion and conducts the assessment 1304. Furthermore, the EMT modifies the identifier by entering additional information gathered from the assessment phase. The serve analyzes the modified identifier and transmits a second suggestion 1305 to a second user portal 1306 at a medical care facility that will be admitting the 9-year-old. A second group of healthcare professionals comprise pediatric nurses and pediatric physicians examine the second suggestion as displayed on the second user portal and begin preparation of properly diagnosing the 9-year-old for the diagnosis phase of the plurality phases of the medical care encounter 1307. The healthcare professionals, after completing the diagnosis, further modify the identifier associated with the 9-year-old by entering additional medical information and lab results at the second user portal. The second user portal relays the entered additional information back 1308) to the server for further analysis. The server generates additional suggestions for other healthcare professionals who are going to perform phases of pre-procedure, procedure, and post-procedure during the course of treating the 9-year-old.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the invention and that methods and structures within the scope of these claims and their equivalents be covered thereby.

1-72. (canceled)

73. A system for treating an individual comprising:
(a) a first non-transitory computer readable medium encoded with first software configured to cause a first processor to:
(i) receive an identifier for said individual;
(ii) associate said identifier with a first phase of a plurality of phases of a medical care encounter experienced by said individual;
(iii) provide, to a first user, a first action to treat said individual during said first phase of said plurality of phases of said medical care encounter experienced by said individual; and
(b) a second non-transitory computer readable medium encoded with second software configured to cause a second processor to:
(i) receive said identifier for said individual;
(ii) associate said identifier with a second phase of a plurality of phases of a medical care encounter experienced by said individual;
(iii) provide, to a second user, a second action to treat said individual during said second phase of said plurality of phases of said medical care encounter experienced by said individual;
wherein said plurality of phases are selected from a group consisting of: an assessment phase, a diagnostic phase, a resuscitation phase, a pre-procedure phase, a procedure phase, a post-procedure phase, critical care phase, after care phase, or discharge.

74. The system of claim 73, wherein said first processor or said second processor is a component of a second computing device.

75. The system of claim 73, wherein said first non-transitory medium or said second non-transitory medium is a component of a remote server.

76. The system of claim 73, wherein said identifier for said individual comprises a name or medical record number of said individual.

77. The system of claim 73, wherein said system further comprises a barcode reader and said software is configured to cause said first processor to receive said identifier from said barcode reader.

78. The system of claim 73, wherein said first user or said second user comprises a healthcare professional selected from the group consisting of a physician, an emergency medical technician, a nurse, a paramedic, a nurse practitioner, a physician's assistant, a nurse's aide, and an x-ray technician.

79. The system of claim 73, wherein said first user or said second user is not a healthcare provider.

80. The system of claim 73, wherein said first action or said second action comprises at least one of a diagnosis, administering therapeutic, administering a dosage of said therapeutic, or a timing of delivery of said therapeutic.

81. The system of claim 73, wherein said first action or said second action comprises a protocol comprising a hospital protocol; a protocol customized to said individual; an evidence-based protocol, or a combination thereof.

82. A computer implemented method for treating an individual, comprising:
(a) receiving an identifier for said individual;
(b) associating said identifier with each phase of a plurality of phases of a medical care encounter experienced by said individual; and
(c) providing, to a user, an action to treat said individual during each phase of said plurality of phases of said medical care encounter experienced by said individual;
wherein said plurality of phases are selected from a group consisting of: an assessment phase, a diagnostic phase, a resuscitation phase, a pre-procedure phase, a procedure phase, a post-procedure phase, critical care phase, after care phase, or discharge.

83. The method of claim 82, wherein said identifier for said individual comprises a name or medical record number of said individual.

84. The method of claim 82, wherein said user comprises a healthcare professional selected from the group consisting of a physician, an emergency medical technician, a nurse, a paramedic, a nurse practitioner, a physician's assistant, a nurse's aide, and an x-ray technician.

85. The method of claim 82, wherein said first action or said second action comprises a diagnosis.

86. The method of claim 82, wherein said action comprises at least one of administering a therapeutic, administering a dosage of said therapeutic, or a timing of delivery of said therapeutic.

87. The method of claim 82, wherein said action comprises a protocol comprising a hospital protocol; a protocol customized to said individual; an evidence-based protocol, or a combination thereof.

88. The method of claim 87, comprising receiving an input from said user indicating that said user completed at least a portion of said protocol using a processor.

89. The method of claim 82, comprising providing to said user a first action in a first phase and a different user a second action in a second phase.

90. The method of claim 82, comprising receiving an input from said first user which at least partially determines said second action.

91. The method of claim 82, wherein said method comprises a software configured to further cause said processor to receive data from an electronic medical record of said individual.

92. The method of claim 82, comprising tracking an outcome of said medical care encounter or whether said action is followed at each of said plurality of phases.
